(19) United States
(12) Patent Application Publication
(10) Pub. No.: US 2026/0286010 A1
(43) Pub. Date: Sep. 24, 2026

(54) TEVp AND KALLIKREIN 6 INHIBITORY NANOBODIES AND METHODS OF USE THEREOF

(71) Applicant: University of Florida Research Foundation, Incorporated, Gainesville, FL (US)

(72) Inventors: Carl A. DENARD, Gainesville, FL (US); Samantha G. MARTINUSEN, Gainesville, FL (US)

(21) Appl. No.: 19/572,099

(22) Filed: Mar. 19, 2026

Related U.S. Application Data
(60) Provisional application No. 63/774,928, filed on Mar. 20, 2025.

(51) Int. Cl. C07K 16/40; C07K 16/10

(52) U.S. Cl. CPC C07K 16/40; C07K 16/10; C07K 2317/33; C07K 2317/565; C07K 2317/569; C07K 2317/76; C07K 2317/92

(57) ABSTRACT

Provided herein are potent orthosteric nanobodies (Nb) and an allosteric inhibitor Nb against tobacco etch virus protease (TEVp) and methods of use thereof. Also provided is a highly selective and potent human kallikrein 6 (KLK6) inhibitory Nb and methods of use thereof.

### CROSS-REFERENCE TO RELATED APPLICATIONS

This application claims the benefit of U.S. Application No. 63/774,928, filed Mar. 20, 2025, which is herein incorporated by reference in its entirety for all purposes.

### GOVERNMENT INTERESTS

This invention was made with government support under Grant No. R21 GM144812 and Grant No. R35 GM146821, awarded by the National Institutes of Health, and Grant No. 2237629, awarded by The National Science Foundation. The government has certain rights in the invention.

### REFERENCE TO A SEQUENCE LISTING SUBMITTED AS AN XML FILE

The Sequence Listing written in file 648103SEQLIST.xml is 84,067 bytes, was created on Mar. 15, 2026, and is hereby incorporated by reference.

### BACKGROUND

Proteases catalyze the hydrolysis of peptide bonds in proteins. Through this reaction, they maintain cellular proteostasis by regulating critical biochemical pathways. Protease dysregulation is a hallmark of various diseases, making them important therapeutic targets in cancer, autoimmune disorders, cardiovascular diseases, neuropathic pain, and infectious diseases. Although proteases represent 5-10% of all drug targets (2010 estimate), only a small fraction of therapeutically relevant proteases (e.g., viral proteases) have been successfully targeted, resulting in only ~20 FDA-approved protease inhibitors. These inhibitors are primarily small molecules and peptidomimetics that engage their target's active site. Unfortunately, these molecules often exhibit poor selectivity and undesirable off-target effects because they cannot differentiate between similar active-site topologies of related proteases. These challenges are exemplified by the failed clinical trials of matrix metalloprotease inhibitors for cancer therapy and BACE1 inhibitors to treat Alzheimer's Disease. While targeting allosteric sites confers higher selectivity, discovering protease allosteric sites and small-molecule allosteric modulators is hampered by an incomplete understanding of the general principles underpinning allosteric and distal regulation. Biased high-throughput screening strategies, residue tethering, activity-based profiling, exosite-targeted screens, and computational methods aim to close this gap. Still, few of these techniques can be applied across all protease families. Taken together, discovery campaigns for selective protease inhibitors remain slow, arduous, and arguably over-reliant on serendipity.

Macromolecular proteinaceous inhibitors can address selectivity and pharmacological limitations faced by small molecules. Particularly, natural protein scaffolds, which include antibodies and their fragments (e.g., nanobodies, scFvs, Fabs), ankyrin repeats, macrocyclic peptides, and cysteine knots can be screened to find exquisitely selective and potent protease inhibitors. By burying more surface area upon binding, macromolecules gain more potency and specificity than small molecules. Furthermore, whereas natural endogenous inhibitors usually do not take advantage of allosteric movements of proteins, protease-inhibitory macromolecules derived from natural scaffolds can attain allosteric regulation by binding to less conserved sequences on the enzyme target and targeting specific conformations. These fine-tuned interactions can elucidate mechanisms that have not been seen in nature and might not have been discovered using traditional high-throughput screening techniques.

There is a critical unmet need to accelerate the discovery and engineering of protease-inhibitory macromolecules. Unfortunately, current selection/screening methods, including hybridoma, phage panning, cell, and bead surface display, coupled with flow cytometry, are primarily based on binding and not inhibition. Thus, extensive downstream characterization is required to isolate inhibitors for large pools of binders. Typically, fewer than 5% of binders isolated through these methods inhibit the targeted protease. Crucially, potentially inhibitory clones with moderate binding affinities are lost during repeated cycles of competitive binding. Previously reported E. coli-based genetic selection for protease inhibitory Fabs and scFvs has a narrow discovery window (isolates many weak inhibitors with >1 μM Kr), requires optimization for each protease target, and may be limited by the activity/toxicity of human proteases in bacteria.

### SUMMARY

The present application introduces HARP (High-throughput Activity Reprogramming of Proteases), a yeast-based strategy that couples the modulation of endoplasmic reticulum (ER) targeted proteases to selectable phenotypes on the yeast cell surface. In this fashion, HARP enables one to discover protease-inhibitory macromolecules from large combinatorial protein binder libraries via fluorescence-activated cell sorting (FACS). Provided herein are potent orthosteric Nbs and an allosteric inhibitor Nb against tobacco etch virus protease (TEVp) and methods of use thereof. Also provided is a highly selective and potent human kallikrein 6 (KLK6) inhibitory Nb and methods of use thereof.

Provided are methods of inhibiting tobacco etch virus protease (TEVp), comprising contacting the TEVp with a nanobody inhibitor. In some such methods, the nanobody inhibitor is selected from the group consisting of SEQ ID NOS: 3, 7, 11, 15, 19-64, and 85-88.

In some such methods, the nanobody inhibitor comprises a sequence at least 95% identical to the sequence set forth in SEQ ID NO: 3. In some such methods, the nanobody inhibitor comprises a sequence at least 95% identical to the sequence set forth in SEQ ID NO: 7. In some such methods, the nanobody inhibitor comprises a sequence at least 95% identical to the sequence set forth in SEQ ID NO: 11. In some such methods, the nanobody inhibitor comprises a sequence at least 95% identical to the sequence set forth in SEQ ID NO: 15. In some such methods, the nanobody inhibitor comprises a sequence at least 95% identical to the sequence set forth in SEQ ID NO: 85. In some such methods, the nanobody inhibitor comprises a sequence at least 95% identical to the sequence set forth in SEQ ID NO: 86. In some such methods, the nanobody inhibitor comprises a sequence at least 95% identical to the sequence set forth in SEQ ID NO: 87. In some such methods, the nanobody inhibitor comprises a sequence at least 95% identical to the sequence set forth in SEQ ID NO: 88.

In some such methods, the nanobody inhibitor comprises the CDR3 sequence set forth in SEQ ID NO: 6. In some such methods, the nanobody inhibitor comprises the CDR3 sequence set forth in SEQ ID NO: 10. In some such methods, the nanobody inhibitor comprises the CDR3 sequence set forth in SEQ ID NO: 14. In some such methods, the nanobody inhibitor comprises the CDR3 sequence set forth in SEQ ID NO: 18.

In some such methods, the nanobody inhibitor has an IC₅₀ of less than 500 nM. In some such methods, the nanobody inhibitor has an IC₅₀ of less than 50 nM. In some such methods, the nanobody inhibitor has a K_{I} of less than 150 nM. In some such methods, the nanobody inhibitor has a K_{I} of less than 25 nM.

Also provided are methods of inhibiting human kallikrein 6 (KLK6), comprising contacting the human KLK6 with a nanobody inhibitor. In some such methods, the contacting occurs within a cancer cell, on the surface of a cancer cell, and/or within a tumor cell microenvironment. In some such methods, the cancer cell and/or tumor cell microenvironment are in a human subject.

In some such methods, the nanobody inhibitor comprises a sequence at least 95% identical to the sequence set forth in SEQ ID NO: 65. In some such methods, the nanobody inhibitor comprises the CDR3 sequence set forth in SEQ ID NO: 68.

In some such methods, the nanobody inhibitor has a K_{D} of less than 500 nM. In some such methods, the nanobody inhibitor has an IC₅₀ of less than 100 nM. In some such methods, the nanobody inhibitor does not inhibit at least 4 other kallikrein family members. In some such methods, the nanobody inhibitor does not inhibit any other kallikrein family members.

### BRIEF DESCRIPTION OF THE FIGURES

FIG. 1 shows an overview of the workflow of HARP. A yeast-based assay of the interaction between a protease, its canonical substrate, and a modulator library within the yeast cell. Quantification of this interaction occurs by fluorescently labeling the displayed substrate cassettes on the surface of the cells, where the desired function (correlating with phenotype) can be selected using fluorescent-activated cell sorting (FACS). Isolated populations are sequenced and purified in preparation for secondary characterization to determine modulator effects and interaction strengths between the modulator and the protease target.

FIG. 2 shows exemplary HARP workflow and resulting FACS output, as well as validation assays with known MMP8 inhibitory and non-inhibitory nanobodies. Panel A shows a schematic representation of the HARP workflow in yeast. Panel B shows Yeast Surface Display (YSD) phenotypes possible using HARP to isolate inhibitors. Panel C shows quantification of observed inhibition of MMP8 as a function of Nb14 ER retention. Panel D shows MMP8 Nanobody (Nb) Inhibition Mock Sort Histograms illustrating the enrichment of protease inhibition (green, anti-HA Alexa 647 signal) going from left to right. Controls included for signal comparison: strong MMP8 Nb inhibitor (red) and non-binder, non-inhibitory Nb (light blue).

FIG. 3 shows synthesis of a large Nb library and isolation of inhibitory Nbs against TEVp. Panel A shows amino acid and length (CDR3) variation present within the NbLibrary_NE_TEVp. Panel B shows a schematic of NbLibrary_NE_TEVp construction prepared for homologous directed recombination (HDR). Complementarity-determining region (CDR) locations are approximate, as well as the endoplasmic reticulum retention signal (if applicable). Panel C shows flow plots for each FACS round of the NbLibrary_NE_TEVp sort with light blue populations corresponding to the positive control (TEVp substrate integrated), red populations corresponding to the negative control (TEVp active proteases integrated), and orange populations corresponding to the NbLibrary_NE_TEVp library populations after each FACS round. Panel D shows the anti-FLAG/anti-HA ratio (normalized to active TEVp protease) of the selected constructs isolated from NbLibrary_NE_TEVp.

FIG. 4 shows NGS analysis of NbLibrary_NE_TEVp. Panel A shows the distribution of CDR3 sequences based on the number of reads in round 4 shows Random-CDR3 with a length of 11 consists of more Unique sequences but with fewer highly repeated CDR3s. Panel B shows a heatmap of amino acid frequency enrichment (Round 4/Naive) for CDR3 sequences. Panel C shows UMAP supervised dimensionality reduction of CDR3 sequence ESM-2 embeddings clustered similar sequences near to each other, for example, VDGDAPYS (SEQ ID NO: 84) is the closest to TEVp-Nb4 CDR3 (VDRGDTGPYP; SEQ ID NO: 6).

FIG. 5 shows sequence alignments of the isolated Nb constructs from the MMP8 mock sort. Amino acid alignment of isolated Nbs from MMP8 Mock Sort RND2 enriched population illustrates all isolated Nbs are identical to the strong inhibitor (Inhibitor sequence, line two of each alignment).

FIG. 6 shows the results of solubility and stability analysis of optimized TEVp nanobodies.

### DETAILED DESCRIPTION

### I. Overview

To address the challenges associated with discovery and engineering of protease-inhibitory macromolecules, we developed HARP (High-throughput Activity Reprogramming of Proteases), a yeast-based strategy that couples the modulation of endoplasmic reticulum (ER) targeted proteases to selectable phenotypes on the yeast cell surface. In this fashion, HARP enables one to discover protease-inhibitory macromolecules from large combinatorial protein binder libraries via fluorescence-activated cell sorting (FACS). Using this platform and an established MMP8 inhibitory nanobody (Nb), we established key principles of the HARP platform to isolate protease inhibitory Nbs. We then demonstrated HARP's function by isolating, for the first time, potent orthosteric Nbs and an allosteric inhibitor Nb against tobacco etch virus protease (TEVp). Deep sequencing of the TEVp Nb library revealed that inhibition phenotypes from yeast correlate linearly with in vitro performance, establishing HARP as a robust and quantitative inhibitor discovery platform. ESM-2 embedding followed by supervised dimensionality reduction of TEVp inhibitory Nb sequences showed distinct clusters among inhibitory Nbs based on their sequence similarities and a clear demarcation between inhibitory and non-inhibitory sequences. We further demonstrated the power of the HARP platform by isolating the first ever highly selective and potent human kallikrein 6 (KLK6) inhibitory Nb.

### II. Nanobody Inhibition of Tobacco Etch Virus Protease (TEVp)

### A. Tobacco Etch Virus Protease (TEVp)

Tobacco etch virus protease (TEVp; UniProt ID: P04517) is a cysteine endopeptidase encoded by the Nuclear Inclusion a (NIa) gene of the tobacco etch virus. It displays stringent substrate specificity and is widely used for biotechnology research, particularly for the processing of fusion proteins, and it has also been engineered for the activation of modified proenzymes. TEVp recognizes the peptide sequence ENLYFQIS/G, where Q and S/G correspond to the P1 and P1′ residues, respectively.

### B. Methods of Nanobody Inhibition of TEVp

Provided are methods of inhibiting tobacco etch virus protease (TEVp), comprising contacting the TEVp with a nanobody inhibitor. As used herein, the term “contacting” refers to placing the nanobody in a location that will allow it to physically interact with TEVp or any variant of TEVp. The contacting may be accomplished using any suitable methods and can occur in vitro, ex vivo, or in vivo. For example, contacting can be accomplished by adding the nanobody to a tube comprising TEVp. Contacting can also be accomplished by adding the nanobody to a medium (e.g., culture medium) comprising cells. These cells can be any type of cell expressing TEVp. Those skilled in the art will recognize that such contacting can be accomplished in any in vitro or in vivo system comprising TEVp, in which the inhibition of TEVp or any variant of TEVp is desired.

In some such methods, the nanobody inhibitor is selected from the group consisting of SEQ ID NOS: 3, 7, 11, 15, 19-64, and 85-88.

In some such methods, the nanobody inhibitor comprises a sequence at least 95% identical to the sequence set forth in SEQ ID NO: 3. In some such methods, the nanobody inhibitor comprises a sequence at least 96% identical to the sequence set forth in SEQ ID NO: 3. In some such methods, the nanobody inhibitor comprises a sequence at least 97% identical to the sequence set forth in SEQ ID NO: 3. In some such methods, the nanobody inhibitor comprises a sequence at least 98% identical to the sequence set forth in SEQ ID NO: 3. In some such methods, the nanobody inhibitor comprises a sequence at least 99% identical to the sequence set forth in SEQ ID NO: 3. In some such methods, the nanobody inhibitor comprises the sequence set forth in SEQ ID NO: 3. In some such methods, the nanobody inhibitor consists essentially of or consists of the sequence set forth in SEQ ID NO: 3.

In some such methods, the nanobody inhibitor comprises a sequence at least 95% identical to the sequence set forth in SEQ ID NO: 7. In some such methods, the nanobody inhibitor comprises a sequence at least 96% identical to the sequence set forth in SEQ ID NO: 7. In some such methods, the nanobody inhibitor comprises a sequence at least 97% identical to the sequence set forth in SEQ ID NO: 7. In some such methods, the nanobody inhibitor comprises a sequence at least 98% identical to the sequence set forth in SEQ ID NO: 7. In some such methods, the nanobody inhibitor comprises a sequence at least 99% identical to the sequence set forth in SEQ ID NO: 7. In some such methods, the nanobody inhibitor comprises the sequence set forth in SEQ ID NO: 7. In some such methods, the nanobody inhibitor consists essentially of or consists of the sequence set forth in SEQ ID NO: 7.

In some such methods, the nanobody inhibitor comprises a sequence at least 95% identical to the sequence set forth in SEQ ID NO: 11. In some such methods, the nanobody inhibitor comprises a sequence at least 96% identical to the sequence set forth in SEQ ID NO: 11. In some such methods, the nanobody inhibitor comprises a sequence at least 97% identical to the sequence set forth in SEQ ID NO: 11. In some such methods, the nanobody inhibitor comprises a sequence at least 98% identical to the sequence set forth in SEQ ID NO: 11. In some such methods, the nanobody inhibitor comprises a sequence at least 99% identical to the sequence set forth in SEQ ID NO: 11. In some such methods, the nanobody inhibitor comprises the sequence set forth in SEQ ID NO: 11. In some such methods, the nanobody inhibitor consists essentially of or consists of the sequence set forth in SEQ ID NO: 11.

In some such methods, the nanobody inhibitor comprises a sequence at least 95% identical to the sequence set forth in SEQ ID NO: 15. In some such methods, the nanobody inhibitor comprises a sequence at least 96% identical to the sequence set forth in SEQ ID NO: 15. In some such methods, the nanobody inhibitor comprises a sequence at least 97% identical to the sequence set forth in SEQ ID NO: 15. In some such methods, the nanobody inhibitor comprises a sequence at least 98% identical to the sequence set forth in SEQ ID NO: 15. In some such methods, the nanobody inhibitor comprises a sequence at least 99% identical to the sequence set forth in SEQ ID NO: 15. In some such methods, the nanobody inhibitor comprises the sequence set forth in SEQ ID NO: 15. In some such methods, the nanobody inhibitor consists essentially of or consists of the sequence set forth in SEQ ID NO: 15.

In some such methods, the nanobody inhibitor comprises a sequence at least 95% identical to the sequence set forth in SEQ ID NO: 85. In some such methods, the nanobody inhibitor comprises a sequence at least 96% identical to the sequence set forth in SEQ ID NO: 85. In some such methods, the nanobody inhibitor comprises a sequence at least 97% identical to the sequence set forth in SEQ ID NO: 85. In some such methods, the nanobody inhibitor comprises a sequence at least 98% identical to the sequence set forth in SEQ ID NO: 85. In some such methods, the nanobody inhibitor comprises a sequence at least 99% identical to the sequence set forth in SEQ ID NO: 85. In some such methods, the nanobody inhibitor comprises the sequence set forth in SEQ ID NO: 85. In some such methods, the nanobody inhibitor consists essentially of or consists of the sequence set forth in SEQ ID NO: 85.

In some such methods, the nanobody inhibitor comprises a sequence at least 95% identical to the sequence set forth in SEQ ID NO: 86. In some such methods, the nanobody inhibitor comprises a sequence at least 96% identical to the sequence set forth in SEQ ID NO: 86. In some such methods, the nanobody inhibitor comprises a sequence at least 97% identical to the sequence set forth in SEQ ID NO: 86. In some such methods, the nanobody inhibitor comprises a sequence at least 98% identical to the sequence set forth in SEQ ID NO: 86. In some such methods, the nanobody inhibitor comprises a sequence at least 99% identical to the sequence set forth in SEQ ID NO: 86. In some such methods, the nanobody inhibitor comprises the sequence set forth in SEQ ID NO: 86. In some such methods, the nanobody inhibitor consists essentially of or consists of the sequence set forth in SEQ ID NO: 86.

In some such methods, the nanobody inhibitor comprises a sequence at least 95% identical to the sequence set forth in SEQ ID NO: 87. In some such methods, the nanobody inhibitor comprises a sequence at least 96% identical to the sequence set forth in SEQ ID NO: 87. In some such methods, the nanobody inhibitor comprises a sequence at least 97% identical to the sequence set forth in SEQ ID NO: 87. In some such methods, the nanobody inhibitor comprises a sequence at least 98% identical to the sequence set forth in SEQ ID NO: 87. In some such methods, the nanobody inhibitor comprises a sequence at least 99% identical to the sequence set forth in SEQ ID NO: 87. In some such methods, the nanobody inhibitor comprises the sequence set forth in SEQ ID NO: 87. In some such methods, the nanobody inhibitor consists essentially of or consists of the sequence set forth in SEQ ID NO: 87.

In some such methods, the nanobody inhibitor comprises a sequence at least 95% identical to the sequence set forth in SEQ ID NO: 88. In some such methods, the nanobody inhibitor comprises a sequence at least 96% identical to the sequence set forth in SEQ ID NO: 88. In some such methods, the nanobody inhibitor comprises a sequence at least 97% identical to the sequence set forth in SEQ ID NO: 88. In some such methods, the nanobody inhibitor comprises a sequence at least 98% identical to the sequence set forth in SEQ ID NO: 88. In some such methods, the nanobody inhibitor comprises a sequence at least 99% identical to the sequence set forth in SEQ ID NO: 88. In some such methods, the nanobody inhibitor comprises the sequence set forth in SEQ ID NO: 88. In some such methods, the nanobody inhibitor consists essentially of or consists of the sequence set forth in SEQ ID NO: 88.

In some such methods, the nanobody inhibitor comprises the CDR3 sequence set forth in SEQ ID NO: 6. In some such methods, the nanobody inhibitor comprises the CDR3 sequence set forth in SEQ ID NO: 10. In some such methods, the nanobody inhibitor comprises the CDR3 sequence set forth in SEQ ID NO: 14. In some such methods, the nanobody inhibitor comprises the CDR3 sequence set forth in SEQ ID NO: 18.

In some such methods, the nanobody inhibitor has an IC₅₀ of less than 500 nM. In some such methods, the nanobody inhibitor has an IC₅₀ of less than 450 nM. In some such methods, the nanobody inhibitor has an IC₅₀ of less than 400 nM. In some such methods, the nanobody inhibitor has an IC₅₀ of less than 350 nM. In some such methods, the nanobody inhibitor has an IC₅₀ of less than 300 nM. In some such methods, the nanobody inhibitor has an IC₅₀ of less than 250 nM. In some such methods, the nanobody inhibitor has an IC₅₀ of less than 200 nM. In some such methods, the nanobody inhibitor has an IC₅₀ of less than 150 nM. In some such methods, the nanobody inhibitor has an IC₅₀ of less than 100 nM. In some such methods, the nanobody inhibitor has an IC₅₀ of less than 90 nM. In some such methods, the nanobody inhibitor has an IC₅₀ of less than 80 nM. In some such methods, the nanobody inhibitor has an IC₅₀ of less than 70 nM. In some such methods, the nanobody inhibitor has an IC₅₀ of less than 60 nM. In some such methods, the nanobody inhibitor has an IC₅₀ of less than 50 nM. In some such methods, the nanobody inhibitor has an IC₅₀ of less than 40 nM. In some such methods, the nanobody inhibitor has an IC₅₀ of less than 30 nM.

In some such methods, the nanobody inhibitor has a K_{I} of less than 150 nM. In some such methods, the nanobody inhibitor has a K_{I} of less than 140 nM. In some such methods, the nanobody inhibitor has a K_{I} of less than 130 nM. In some such methods, the nanobody inhibitor has a K_{I} of less than 120 nM. In some such methods, the nanobody inhibitor has a K_{I} of less than 110 nM. In some such methods, the nanobody inhibitor has a K_{I} of less than 100 nM. In some such methods, the nanobody inhibitor has a K_{I} of less than 90 nM. In some such methods, the nanobody inhibitor has a K_{I} of less than 80 nM. In some such methods, the nanobody inhibitor has a K_{I} of less than 70 nM. In some such methods, the nanobody inhibitor has a K_{I} of less than 60 nM. In some such methods, the nanobody inhibitor has a K_{I} of less than 50 nM. In some such methods, the nanobody inhibitor has a K_{I} of less than 40 nM. In some such methods, the nanobody inhibitor has a K_{I} of less than 30 nM. In some such methods, the nanobody inhibitor has a K_{I} of less than 25 nM. In some such methods, the nanobody inhibitor has a K_{I} of less than 20 nM. In some such methods, the nanobody inhibitor has a K_{I} of less than 15 nM. In some such methods, the nanobody inhibitor has a Kr of less than 10 nM.

### III. Nanobody Inhibition of Human Kallikrein 6 (KLK6)

### A. Human Kallikrein 6 (KLK6)

Human tissue kallikreins (hKs) comprise a subgroup of 15 homologous secreted trypsin or chymotrypsin-like serine proteases, encoded by a tightly clustered multigene family on chromosome 19q13.4. hKs are primarily expressed within the glandular epithelia of many organs and implicated in a range of normal physiological functions. However, hK genes/proteins are aberrantly expressed in many cancer types, and their expression is often associated with patient prognosis.

Among hKs, human kallikrein 6 (KLK6 or hK6) (NCBI Gene ID: 5653; UniProt ID: Q92876) is overexpressed in several cancers, driving tumor invasion and metastasis. hK6 may also be a viable target to combat neurodegeneration.

### B. Methods of Nanobody Inhibition of KLK6

Provided are methods of inhibiting human kallikrein 6 (KLK6), comprising contacting the human KLK6 with a nanobody inhibitor. As used herein, the term “contacting” refers to placing the nanobody in a location that will allow it to physically interact with KLK6 or any variant of KLK6. The contacting may be accomplished using any suitable methods and can occur in vitro, ex vivo, or in vivo. In some such methods, the contacting occurs within a cancer cell, on the surface of a cancer cell, and/or within a tumor cell microenvironment. As used herein, “tumor cell microenvironment” refers to the area surrounding a tumor (including the tumor itself), comprising cancer cells, immune cells, stromal cells, blood vessels, and extracellular matrix (ECM).

The cancer cell can be any type of cancer cell. For example, the cancer cell can be a breast cancer cell, a prostate cancer cell, an ovarian cancer cell, a uterine cancer cell, a lung cancer cell, a colorectal cancer cell, a liver cancer cell, a kidney cancer cell, a brain cancer cell, a thyroid cancer cell, a bone cancer cell or other sarcoma cell, or a lymphoma cell. In some such methods, the cancer cell is a breast cancer cell. In some such methods, the cancer cell is a prostate cancer cell. In some such methods, the cancer cell is an ovarian cancer cell. In some such methods, the cancer cell is a uterine cancer cell. In some such methods, the cancer cell is a lung cancer cell. In some such methods, the cancer cell is a colorectal cancer cell. In some such methods, the cancer cell is a liver cancer cell. In some such methods, the cancer cell is a kidney cancer cell. In some such methods, the cancer cell is a brain cancer cell. In some such methods, the cancer cell is a thyroid cancer cell. In some such methods, the cancer cell is a bone cancer cell or other sarcoma cell. In some such methods, the cancer cell is a lymphoma cell.

In some such methods, the cancer cell is in vitro. In some such methods, the cancer cell and/or tumor cell microenvironment is in vivo. In some such methods, the cancer cell and/or tumor cell microenvironment are in a human subject.

In the case of contacting KLK6 in a human subject, the nanobody inhibitor can be administered in a pharmaceutical composition. Such pharmaceutical compositions can include a therapeutically effective amount of an active ingredient (e.g., nanobody inhibitor) and at least one pharmaceutically acceptable carrier (e.g., adjuvant or diluent). The carrier in the pharmaceutical compositions must be “acceptable” in the sense that it is compatible with the active ingredient of the composition (and preferably, capable of stabilizing the active ingredient) and not deleterious to the patient to be treated. One or more solubilizing agents can be utilized as pharmaceutical carriers for delivery of the active ingredients described herein. Examples of other carriers include colloidal silicon oxide, magnesium stearate, cellulose, sodium lauryl sulfate, and D&C Yellow #10. The pharmaceutical composition can optionally include at least one further additive selected from a disintegrating agent, binder, lubricant, flavoring agent, preservative, colorant and any mixture thereof. Examples of such and other additives can be found in “Handbook of Pharmaceutical Excipients”; Ed. A. H. Kibbe, 3rd Ed., American Pharmaceutical Association, USA and Pharmaceutical Press UK, 2000.

A pharmaceutical composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

The pharmaceutical compositions described herein can be adapted for parenteral, oral, topical, nasal, rectal, buccal, or sublingual administration or for administration via the respiratory tract, e.g., in the form of an aerosol or an air-suspended fine powder. The term “parenteral” as used herein refers to subcutaneous, intracutaneous, intravenous, intramuscular, intraarticular, intraarterial, intrasynovial, intrasternal, intrathecal, intralesional, intraperitoneal, intraocular, intra-aural, or intracranial injection, as well as any suitable infusion technique. The pharmaceutical composition can be in the form of tablets, capsules, powders, microparticles, granules, syrups, suspensions, solutions, nasal spray, transdermal patches, injectable solutions, or suppositories.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

Administration of the pharmaceutical composition to a human subject to accomplish contacting KLK6 with the nanobody inhibitor can be performed once daily, twice daily, three times daily, four times daily, or more. Administration can be performed for any required length of time, repeating daily, weekly, monthly, or any other interval (e.g., alternating days, bi-weekly, etc.) to achieve the desired effect.

In some such methods, the nanobody inhibitor comprises a sequence at least 95% identical to the sequence set forth in SEQ ID NO: 65. In some such methods, the nanobody inhibitor comprises a sequence at least 96% identical to the sequence set forth in SEQ ID NO: 65. In some such methods, the nanobody inhibitor comprises a sequence at least 97% identical to the sequence set forth in SEQ ID NO: 65. In some such methods, the nanobody inhibitor comprises a sequence at least 98% identical to the sequence set forth in SEQ ID NO: 65. In some such methods, the nanobody inhibitor comprises a sequence at least 99% identical to the sequence set forth in SEQ ID NO: 65. In some such methods, the nanobody inhibitor comprises the sequence set forth in SEQ ID NO: 65. In some such methods, the nanobody inhibitor consists essentially of or consists of the sequence set forth in SEQ ID NO: 65. In some such methods, the nanobody inhibitor comprises the CDR3 sequence set forth in SEQ ID NO: 68.

In some such methods, the nanobody inhibitor has a K_{D} of less than 500 nM. In some such methods, the nanobody inhibitor has a K_{D} of less than 450 nM. In some such methods, the nanobody inhibitor has a K_{D} of less than 400 nM. In some such methods, the nanobody inhibitor has a K^{D} of less than 350 nM. In some such methods, the nanobody inhibitor has an IC₅₀ of less than 150 nM. In some such methods, the nanobody inhibitor has an IC₅₀ of less than 140 nM. In some such methods, the nanobody inhibitor has an IC₅₀ of less than 130 nM. In some such methods, the nanobody inhibitor has an IC₅₀ of less than 120 nM. In some such methods, the nanobody inhibitor has an IC₅₀ of less than 110 nM. In some such methods, the nanobody inhibitor has an IC₅₀ of less than 100 nM. In some such methods, the nanobody inhibitor has an IC₅₀ of less than 95 nM. In some such methods, the nanobody inhibitor has an IC₅₀ of less than 90 nM.

In some such methods, the nanobody inhibitor does not inhibit at least 1 other kallikrein family member. In some such methods, the nanobody inhibitor does not inhibit at least 2 other kallikrein family members. In some such methods, the nanobody inhibitor does not inhibit at least 3 other kallikrein family members. In some such methods, the nanobody inhibitor does not inhibit at least 4 other kallikrein family members. In some such methods, the nanobody inhibitor does not inhibit at least 5 other kallikrein family members. In some such methods, the nanobody inhibitor does not inhibit at least 6 other kallikrein family members. In some such methods, the nanobody inhibitor does not inhibit at least 7 other kallikrein family members. In some such methods, the nanobody inhibitor does not inhibit at least 8 other kallikrein family members. In some such methods, the nanobody inhibitor does not inhibit at least 9 other kallikrein family members. In some such methods, the nanobody inhibitor does not inhibit at least 10 other kallikrein family members. In some such methods, the nanobody inhibitor does not inhibit at least 11 other kallikrein family members. In some such methods, the nanobody inhibitor does not inhibit at least 12 other kallikrein family members. In some such methods, the nanobody inhibitor does not inhibit at least 13 other kallikrein family members. In some such methods, the nanobody inhibitor does not inhibit at least 14 other kallikrein family members. In some such methods, the nanobody inhibitor does not inhibit any other kallikrein family members.

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

In general, the term “about” indicates variation in a quantity of a component of a composition not having a significant effect on the activity or stability of the composition. For example, “about” can mean within 1 standard deviation. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15.

The term “and/or” refers to and encompasses any and all possible combinations of one or more of the associated listed items, as well as the lack of combinations when interpreted in the alternative (“or”)

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. In the event of a conflict between a sequence in the application and an indicated accession number or position in an accession number, the sequence in the application predominates. The effective filing date means the earlier of the actual filing date or filing date of a priority application referring to the accession number, if applicable. Likewise, if different versions of a publication, website, or the like are published at different times, the version most recently published at the effective filing date of the application is meant, unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases and one letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 1
Description of Sequences

| SEQ ID NO | Type | Description |
|---|---|---|
| 1 | Protein | ER retention signal |
| 2 | Protein | ER retention signal |
| 3 | Protein | TEVp-Nb4 |
| 4 | Protein | TEVp-Nb4 CDR1 |
| 5 | Protein | TEVp-Nb4 CDR2 |
| 6 | Protein | TEVp-Nb4 CDR3 |
| 7 | Protein | TEVp-Nb9 |
| 8 | Protein | TEVp-Nb9 CDR1 |
| 9 | Protein | TEVp-Nb9 CDR2 |
| 10 | Protein | TEVp-Nb9 CDR3 |
| 11 | Protein | TEVp-Nb5 |
| 12 | Protein | TEVp-Nb5 CDR1 |
| 13 | Protein | TEVp-Nb5 CDR2 |
| 14 | Protein | TEVp-Nb5 CDR3 |
| 15 | Protein | TEVp-Nb7 |
| 16 | Protein | TEVp-Nb7 CDR1 |
| 17 | Protein | TEVp-Nb7 CDR2 |
| 18 | Protein | TEVp-Nb7 CDR3 |
| 19 | Protein | TEVp-Nb Rank 1 |
| 20 | Protein | TEVp-Nb Rank 2 |
| 21 | Protein | TEVp-Nb Rank 4 |
| 22 | Protein | TEVp-Nb Rank 5 |
| 23 | Protein | TEVp-Nb Rank 6 |
| 24 | Protein | TEVp-Nb Rank 7 |
| 25 | Protein | TEVp-Nb Rank 8 |
| 26 | Protein | TEVp-Nb Rank 9 |
| 27 | Protein | TEVp-Nb Rank 10 |
| 28 | Protein | TEVp-Nb Rank 11 |
| 29 | Protein | TEVp-Nb Rank 12 |
| 30 | Protein | TEVp-Nb Rank 14 |
| 31 | Protein | TEVp-Nb Rank 15 |
| 32 | Protein | TEVp-Nb Rank 16 |
| 33 | Protein | TEVp-Nb Rank 17 |
| 34 | Protein | TEVp-Nb Rank 18 |
| 35 | Protein | TEVp-Nb Rank 19 |
| 36 | Protein | TEVp-Nb Rank 20 |
| 37 | Protein | TEVp-Nb Rank 21 |
| 38 | Protein | TEVp-Nb Rank 22 |
| 39 | Protein | TEVp-Nb Rank 24 |
| 40 | Protein | TEVp-Nb Rank 25 |
| 41 | Protein | TEVp-Nb Rank 26 |
| 42 | Protein | TEVp-Nb Rank 27 |
| 43 | Protein | TEVp-Nb Rank 28 |
| 44 | Protein | TEVp-Nb Rank 29 |
| 45 | Protein | TEVp-Nb Rank 30 |
| 46 | Protein | TEVp-Nb Rank 31 |
| 47 | Protein | TEVp-Nb Rank 32 |
| 48 | Protein | TEVp-Nb Rank 33 |
| 49 | Protein | TEVp-Nb Rank 34 |
| 50 | Protein | TEVp-Nb Rank 35 |
| 51 | Protein | TEVp-Nb Rank 36 |
| 52 | Protein | TEVp-Nb Rank 37 |
| 53 | Protein | TEVp-Nb Rank 38 |
| 54 | Protein | TEVp-Nb Rank 39 |
| 55 | Protein | TEVp-Nb Rank 40 |
| 56 | Protein | TEVp-Nb Rank 41 |
| 57 | Protein | TEVp-Nb Rank 42 |
| 58 | Protein | TEVp-Nb Rank 43 |
| 59 | Protein | TEVp-Nb Rank 44 |
| 60 | Protein | TEVp-Nb Rank 45 |
| 61 | Protein | TEVp-Nb Rank 46 |
| 62 | Protein | TEVp-Nb Rank 47 |
| 63 | Protein | TEVp-Nb Rank 48 |
| 64 | Protein | TEVp-Nb Rank 50 |
| 65 | Protein | hK6-Nb1 |
| 66 | Protein | hK6-Nb1 CDR1 |
| 67 | Protein | hK6-Nb1 CDR2 |
| 68 | Protein | hK6-Nb1 CDR3 |
| 69 | Protein | hK6-Nb3 |
| 70 | Protein | hK6-Nb7 |
| 71 | DNA | TWIST-NbLib-Nested-F |
| 72 | DNA | TWIST-NbLib-Nested-R |
| 73 | DNA | Making-NbLib-F |
| 74 | DNA | Making NbLib-R |
| | | (contains FEHDEL [SEQ |
| | | ID NO: 2] ERS) |
| 75 | DNA | Making NbLib-R |
| | | (without ERS) |
| 76 | DNA | BsaI-NbLib-NE-PCR-F |
| 77 | DNA | BsaI-NbLib-NE-PCR-R |
| 78 | Protein | Canonical TEVp substrate |
| 79 | Protein | TEVp cleavage product |
| 80 | Protein | FRET peptide |
| 81 | Protein | FRET peptide |
| 82 | Protein | MMP8 non-inhibitor Nb |
| 83 | Protein | MMP inhibitor Nb |
| 84 | Protein | UMAP clustered CDR3 |
| 85 | Protein | Optimized TEVp DNB_10 |
| 86 | Protein | Optimized TEVp DNB_11 |
| 87 | Protein | Optimized TEVp DNB_19 |
| 88 | Protein | Optimized TEVp DNB_22 |

### EXAMPLES

### Example 1. Proof of Concept Validation of HARP System with an Established MMP8 Inhibitory Nanobody

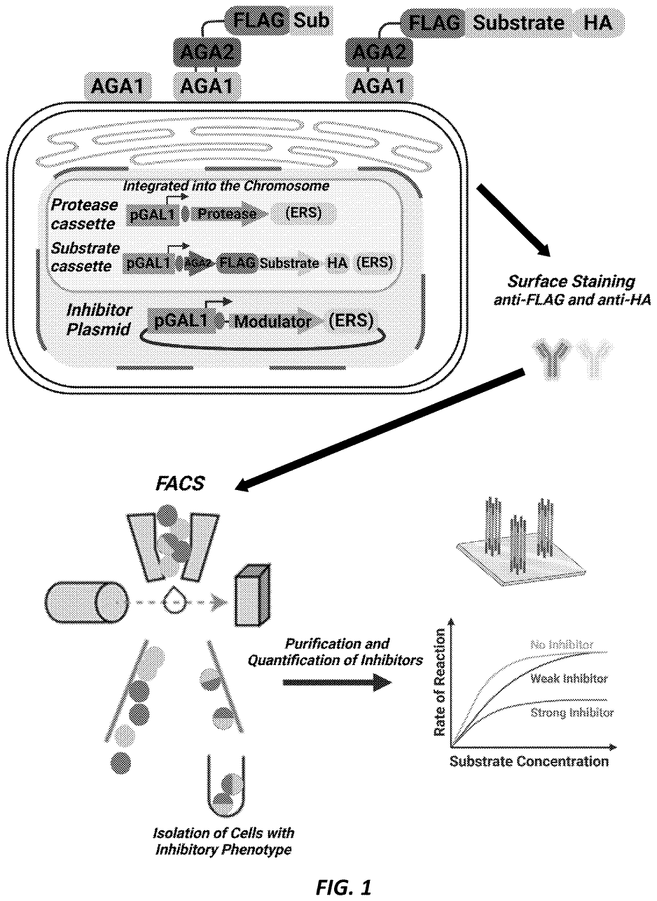

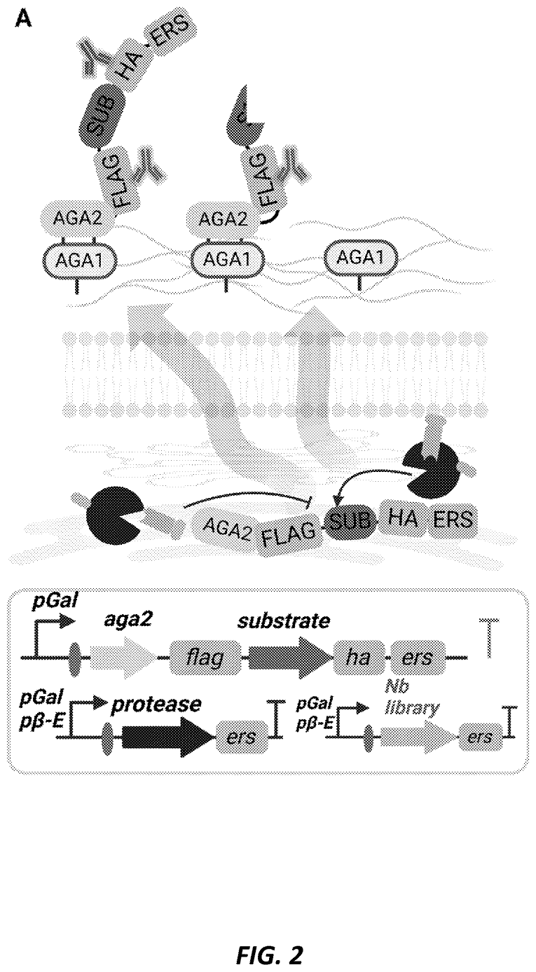

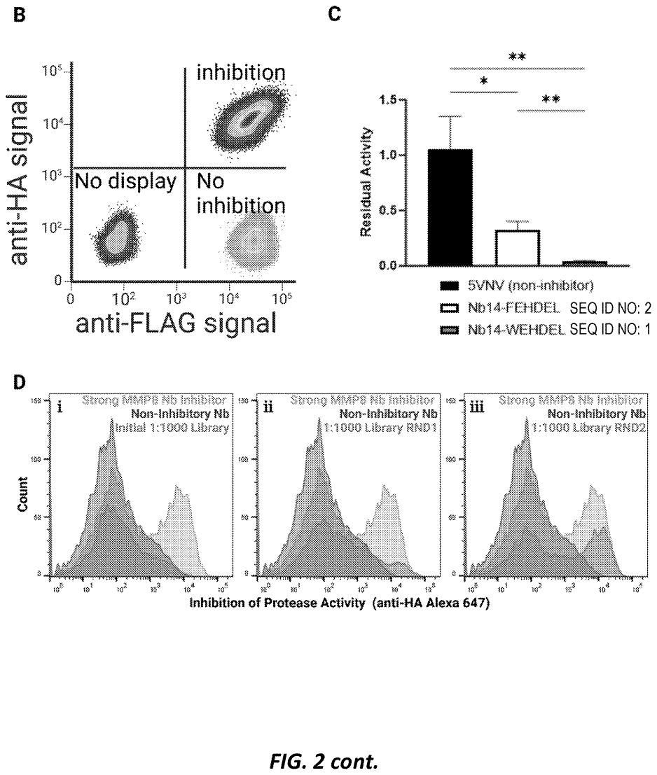

ER sequestration and screening, empowered by powerful transcriptional and post-translational strategies, has proven instrumental for the high-throughput interrogation and engineering of post-translationally modified enzymes' activities, including proteases, kinases, and methyltransferases. We reasoned that one could leverage this approach to isolate genetically encoded macromolecules that modulate the activity of ER-sequestered proteases, for example, through their inhibition. Thus, we set out to build the HARP system (FIG. 1), a high-throughput functional screen for protease modulatory macromolecule discovery and engineering. HARP consists of three transcriptional units: a protease, a substrate cassette, and a protein-based modulator (FIG. 2, Panel A). The protease contains a 5′ ER targeting sequence and a 3′ ER retention signal, functionally immobilizing it in the yeast ER. Similarly, the protein-based protease modulator is targeted to the ER and may contain an ER retention signal (ERS) to increase its ER concentration and interaction time with the protease. Finally, the substrate cassette, which reports the activity of the ER-resident protease, is an AGA2 fusion polypeptide that transits through the ER on its way to the cell surface. Substrate ER residence time can be manipulated by appending an ERS at the 3′ end of the AGA2-substrate cassette. The AGA2-substrate polypeptide fusion contains unique epitope tags flanking the substrate sequence that can be stained with fluorescently labeled antibodies once on the cell surface. Thus, this substrate cassette serves as an extracellular reporter of enzyme activities. During the operation of the HARP platform with a protease, three phenotypes are possible: (1) no surface display, (2) a cleaved substrate (resulting from the protease remaining active), and (3) an intact substrate (resulting from an inhibited protease or a substrate-targeted inhibitor) (FIG. 2, Panel B). Cells displaying the desired phenotype (in this case, an intact substrate) can be selected and enriched using fluorescence-activated cell sorting (FACS).

To demonstrate functionality of the HARP system, we conducted a proof-of-concept experiment using a published MMP8 inhibitory nanobody (Nb), Nb14. Nb14, discovered from a phage display Nb library, has a dissociation constant (K_{D}) of 1.334±0.137 nM but a poor half maximal inhibitory concentration (IC₅₀) of 4.359±0.237 μM⁴¹. This highlights a common issue where strong enzyme binders selected through display methods often function as weak inhibitors. To evaluate Nb14-mediated MMP8 inhibition in yeast, we co-expressed Nb14 and MMP8 in the yeast ER with an ER-transiting AGA2-MMP8 substrate fusion. MMP8 and its substrate contained a C-terminal WEHDEL (SEQ ID NO: 1) ERS to set a high MMP8 activity in the ER. MMP8 expression was controlled by a β-estradiol-inducible promoter to avoid cytotoxicity observed with pGAL1-driven expression. As a control, Nb14 was replaced with the non-binding, non-inhibitory Nb, 5vnv. As expected, 5vnv showed no inhibition of MMP8 activity, and Nb14's inhibitory potency depended on its C-terminal ER retention signal (ERS) strength. The WEHDEL (SEQ ID NO: 1) ERS inhibited MMP8 by 96%, while the FEHDEL (SEQ ID NO: 2) ERS reduced MMP8 activity by 67% (FIG. 2, Panel C).

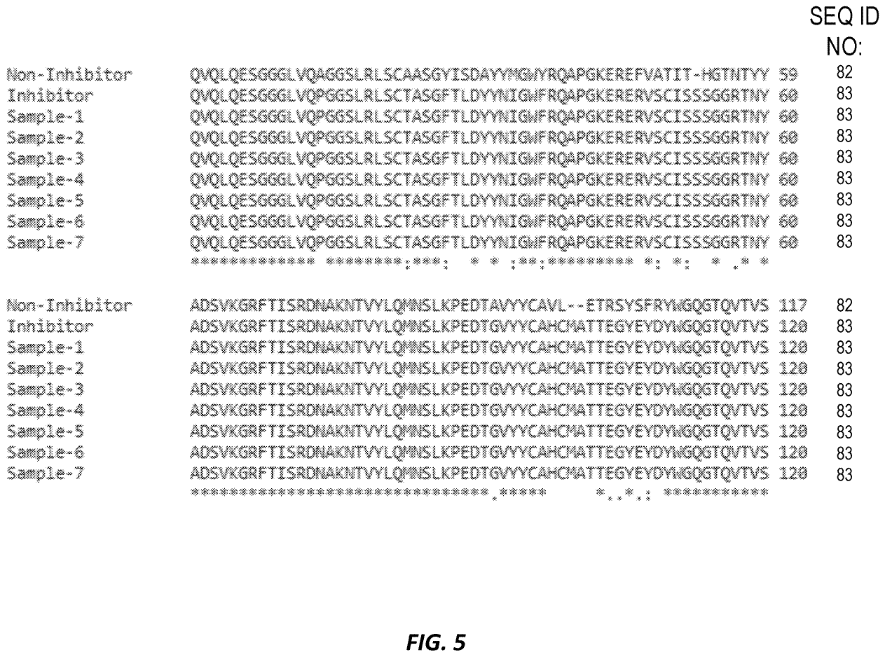

Next, we performed a mock sort to evaluate HARP's ability to isolate inhibitory proteins from a large pool of non-inhibitors. We mixed cells expressing Nb14/MMP8/MMP8 substrate with cells expressing 5vnv/MMP8/MMP8 substrate at a 1:1000 ratio. All constructs included a strong C-terminal WEHDEL (SEQ ID NO: 1) ERS. After three rounds of sorting for an inhibitory phenotype, indicated by high anti-FLAG and high anti-HA signals, the library was fully enriched for Nb14-expressing cells, verified by Sanger sequencing of DNA isolated from ten isolated clones (data not shown). This result demonstrated HARP's capability to effectively identify inhibitory proteins from a large, diverse population (FIG. 5).

### Example 2. High-Throughput Discovery of TEVp Inhibitory Nanobodies

Having established HARP's screening capability, we next isolated protease inhibitory nanobodies from a large synthetic library, choosing tobacco etch virus protease (TEVp) as a test case. We selected nanobodies (Nbs) as our initial inhibitory scaffold because their longer complementarity-determining regions (CDRs) (average length of 15 amino acids) can adopt convex paratope structures that allow them to penetrate enzyme active sites. Nb CDRs can also engage distal pockets or surface loops on their targets that could confer allostery. Lastly, since Nbs are relatively small (15 kDa) and can be easily expressed in the periplasm of E. coli, they fit within our streamlined in vitro characterization pipeline. Two TEVp inhibitors have been reported to date. Both are cyclic peptides chemically modified with a covalent warhead, with IC₅₀ values of 700 nM and 80 nM. Notably, the covalent warhead accounts for the majority of their inhibitory potency. Fully recombinant protease inhibitory Nbs are advantageous over chemically modified macromolecules because they are easier to scale and translate in vivo.

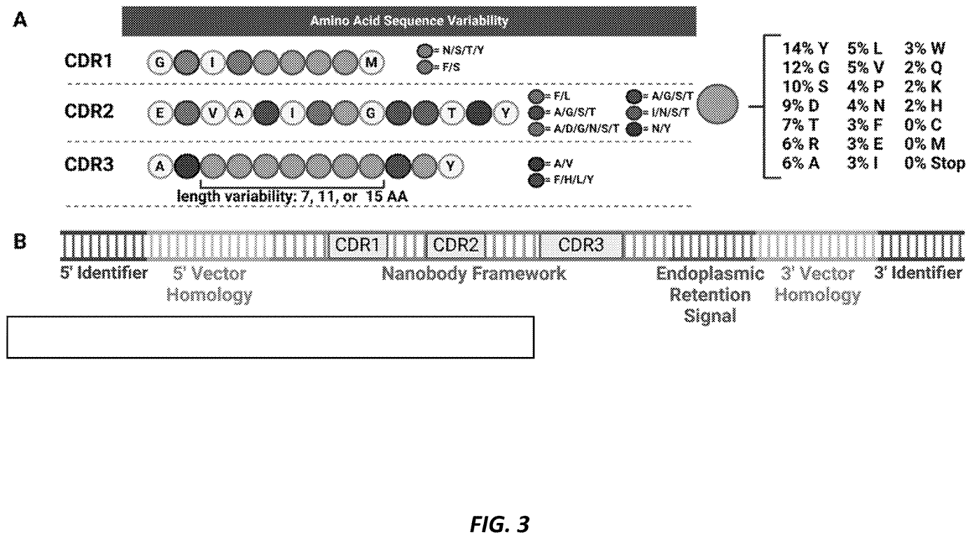

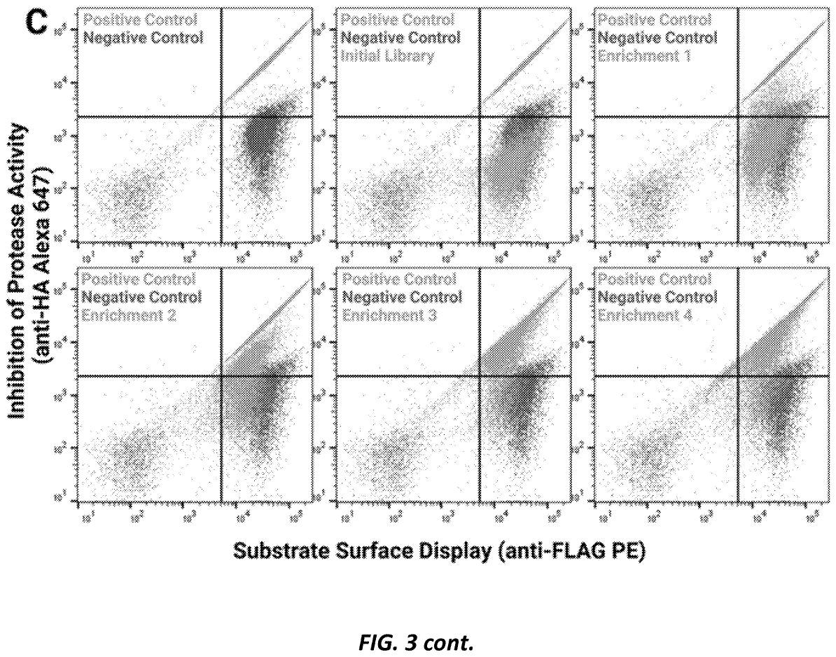

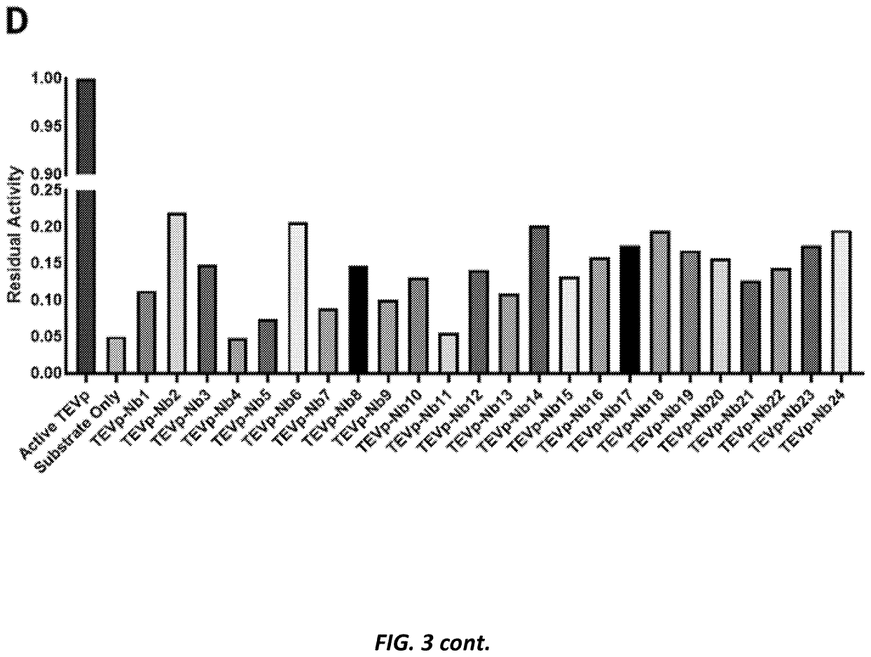

For the inhibitor isolation campaign, we emulated the synthetic camelid Nb library previously reported by McMahon et al. (2018) Nature Structural & Molecular Biology 25:289-296. This 1-billion-member library, built on a curated set of highly stable and biochemically well-behaved Nb structures from the Protein Data Bank (PDB), contains CDRs varying in amino acid composition and CDR3 length (10,14, and 18 amino acids) (FIG. 3, Panels A and B). The 10 amino acid CDR3 comprises 7 completely randomized positions (herein called CDR-7), the 14 amino acid CDR3 has 11 randomized positions (CDR-11), and the 18 amino acid CDR3 has 15 randomized positions (CDR-15). In an initial attempt to discover TEVp inhibitory Nbs, an FEHDEL (SEQ ID NO: 2) ERS was appended to the C-terminus of the Nb library and transformed using homology-directed recombination (HDR) into a yeast strain containing a chromosomally integrated TEVp and TEVp substrate. Nbs isolated from this configuration showed little to no TEVp inhibition in vitro. Moreover, these Nbs failed to retain their inhibitory phenotype in yeast when retested without an ERS (data not shown). This suggested that artificially enhancing Nb::protease contact time in the ER leads to false positives because Nbs with ERS can outcompete the protease for ERS receptors Erd1 and Erd2.

These results highlighted two key considerations for successfully isolating potent inhibitors. First, high enzyme activity (reflected by an anti-FLAG/anti-HA signal normalized ratio of 1) (FIG. 2, Panel C) creates a sufficiently large inhibitor selection window. Using strong promoters to drive protease and substrate expression and appending strong ERSs to both cassettes satisfy this criterion. Second, removing the ERS from the potential inhibitor cassettes can eliminate weak inhibitors during screening. The steady-state ER concentration of an inhibitor population lacking an ERS is lower than that of the protease, as most potential modulators translocate to the cytoplasm. Therefore, while weak and non-binders are “washed out” of the ER, moderate to strong binders remain. However, being a functional screen, HARP only isolates binders that inhibit enzyme activity via FACS. Thus, setting a high protease activity (strong ERS) and reduced inhibitor concentration (no ERS) in HARP applies selective pressure to enrich for potent inhibitors. Therefore, we reformatted the dsDNA Nb library, this time devoid of an ERS, to generate NbLib_TEVp-NE library, comprising approximately 2×10⁷ variants.

To enrich for TEVp inhibitors, cells harboring the NbLib_TEVp-NE (10⁸ cells) library were induced in galactose media and stained with fluorescently labeled antibodies—anti-HA Alexa 647 (BioLegend, cat #682404) and anti-FLAG PE (BioLegend, cat #637309). The stained cells were then subjected to fluorescence-activated cell sorting (FACS). In every round, two control strains were included to guide the gating strategy: (i) a substrate display-only control lacking TEVp (No TEVp), and (ii) the TEVp reporter strain. The goal was to isolate Nb-harboring cells phenotypically resembling those of the substrate display-only control, indicative of potent TEVp inhibition. Thus, gating stringencies were increased from round to round. After four rounds of FACS, during which the NbLib_TEVp-NE library became progressively enriched for cells exhibiting an inhibitory phenotype (populating in the upper-right quadrant), beginning with ~1% (Initial Library) and enriching to ~60% (ENR4) of the total population (FIG. 3, Panel C). At the end of the Nb discovery campaign (ENR4), 24 individual clones were sequenced and rescreened in yeast to evaluate the diversity of inhibitory phenotypes (FIG. 3, Panel D). All clones harbored Nbs that inhibited TEVp by ≥80%.

### In Vitro Characterization of TEVp Inhibitory Nbs

Following single clone screening, we characterized four Nb clones with varying percent residual activity (PRA) in yeast: TEVp-Nb4 (4.8%), TEVp-Nb5 (7.4%), TEVp-Nb7 (8.9%), and TEVp-Nb9 (10%) (FIG. 3, Panel D) to determine whether inhibitory phenotypes observed in yeast correlated with in vitro potencies. Nb half-maximum inhibitory constants (IC₅₀) and inhibition constants (K_{I}) were determined using an Abz-TENLYFQSGK(Dnp) (SEQ ID NO: 80) FRET peptide and are summarized in Table 2. TEVp-Nb4, which had the highest PRA in yeast was the strongest inhibitor in vitro, with an IC₅₀ and K_{I} of 29 and 8 nM, respectively. This makes TEVp-Nb4 the most potent TEVp inhibitor to date. Furthermore, TEVp-Nb5 (PRA: 7.4%, IC₅₀: 201 nM, K_{I}: 53 nM), TEVp-Nb7 (PRA, 8.9%, IC₅₀: 420 nM, K_{I}: 110 nM), and TEVp-Nb9 (PRA: 10%, IC₅₀: 178 nM, K_{I}: 47 nM) all show significant inhibitory potencies (Table 2).

TABLE 2
Inhibition potency characterization of isolated TEVp-Nbs

| | | | | # of |
|---|---|---|---|---|
| | | | | occurrences |
| | | | | in |
| Nb | Sequence | IC₅₀ [nM] | K_{I} [nM] | ENR4 NGS |
| TEVp- | QVQLQESGGGLVQAGGSLRLSCAASGTIFHGQFMG | 29.1 ± 5.4 | 7.6 ± 0.7 | 682 |
| Nb4 | WYRQAPGKERELVATIGSGATTYYADSVKGCFTISRDN | | | |
| | AKNTVYLQMNSLKPEDTAVYYCAVDRGDTGPYPYW | | | |
| | GQGTQVTVSS (SEQ ID NO: 3) | | | |
| | (CDR1-SEQ ID NO: 4; | | | |
| | CDR2-SEQ ID NO: 5; CDR3-SEQ ID NO: 6) | | | |
| TEVp- | QVQLQESGGGLVQAGGSLRLSCAASGTISIYQAMGW | 178.2 ± 76.9 | 46.7 ± 1.6 | 211 |
| Nb9 | YRQAPGKERELVAAIAEGANTNYADSVKGRFTISRDNA | | | |
| | KNTVYLQMNSLKPEDTAVYYCAVAPFYEWYFDYYYI | | | |
| | YWGQGTQVTVSS (SEQ ID NO: 7) | | | |
| | (CDR1-SEQ ID NO: 8; | | | |
| | CDR2-SEQ ID NO: 9; CDR3-SEQ ID NO: 10) | | | |
| TEVp- | QVQLQESGGGLVQAGGSLRLSCAASGYISDPVAMGW | 201 ± 169.9 | 52.7 ± 3.2 | 90 |
| Nb5 | YRQAPGKELELVAAIDYGTTTNYADSVKGRFTISRDNA | | | |
| | KNTVYLQMNSLKPEDTAVYYCAVEAADYLFGYGYY | | | |
| | AYWGQGTQVTVSS (SEQ ID NO: 11) | | | |
| | (CDR1-SEQ ID NO: 12; CDR2-SEQ ID NO: 13; | | | |
| | CDR3-SEQ ID NO: 14) | | | |
| TEVp- | QVQLQESGGGLVQAGGSLRLSCAASGNISSDDLMGW | 419.7 ± 122.9 | 109.9 ± 1.1 | 41 |
| Nb7 | YRQAPGKERELVAAIDTGASTYYADSVKGRFTISCDNA | | | |
| | KNTVYLQMNSLKPEDTAVYYCAAYTYSQDYWGVLT | | | |
| | YVGHGYWGQGTQVTVSS (SEQ ID NO: 15) (CDR1- | | | |
| | SEQ ID NO: 16; CDR2-SEQ ID NO: 17; | | | |
| | CDR3-SEQ ID NO: 18) | | | |

CDR1, CDR2, and CDR3 are indicated by bold, italics, and underline, respectively.

Nb in vitro potencies are generally correlated with their in-yeast performance. For example, the strongest inhibitor, TEVp-Nb4, showed the highest in-yeast performance, and TEVp-Nb7, which showed lower in-yeast performance, had a lower potency in vitro. Therefore, we wondered whether HARP could provide a robust and quantitative measurement of protease inhibition, where Nbs with the highest read counts at the end of the campaign would likely be more inhibitory in vitro. To test this hypothesis, we sequenced the post-round two libraries (26,604 unique sequences) and the final-round library (16,006 unique sequences) from the discovery campaign and determined the number of occurrences for all unique sequences in the final round. Above all, the four Nbs randomly selected for in vitro characterization ranked among the top 50 most abundant sequences (Table 3). The K_{I} values of these four select Nbs corresponded to their positions in the NGS dataset: TEVp-Nb4 (K_{I}=7.6 nM, 3^{rd} most abundant, 682 occurrences), TEVp-Nb9 (K_{I}=46.7 nM, 13^{th} position, 211 occurrences), TEVp-Nb5 (K_{I}=52.7 nM, 23^{rd} position, 90 occurrences), and TEVp-Nb7 (K_{I}=109.9 nM, 48^{th} position, 41 occurrences) (Table 3). Furthermore, the linear relationship between read count, in vitro potency, and, by extension, in-yeast performance carried over to the remaining unique clones (data not shown).

TABLE 3
Read counts of top 50 TEVp-Nb sequences in ENR4.

| | | SEQ | | |
|---|---|---|---|---|
| | | ID | # of | |
| Rank | NB Sequensce | NO | occurrences | * |
| 1 | QVQLQESGGGLVQAGGSLRLSCAASGSIFTDTYMGWYRQAPGKE | 19 | 872 | |
| | RELVATITPGSSTNYADSVKGRFTISRDNAKNTVYLQMNSLKPEDT | | | |
| | AVYYCAAYTYVENQGYYYFAYWGQGTQVTVSS | | | |
| 2 | QVQLQESGGGLVQAGGSLRLSCAASGYIFGGGPMGWYRQAPGKE | 20 | 793 | |
| | REFVATIDPGGITYYADSVKGRFTISRDNAKNTVYLQMNSLKPEDT | | | |
| | AVYYCAAEYTEVDFGSNYWSYGHFYWGQGTQVTVSS | | | |
| 3 | QVQLQESGGGLVQAGGSLRLSCAASGTIFHGQFMGWYRQAPGKE | 3 | 682 | TEVp- |
| | RELVATIGSGATTYYADSVKGCFTISRDNAKNTVYLQMNSLKPED | | | Nb4 |
| | TAVYYCAVDRGDTGPYPYWGQGTQVTVSS | | | |
| 4 | QVQLQESGGGLVQAGDSLRLSCAASGSIFSYY AMGWYRQAPGKE | 21 | 655 | |
| | REFVAGIANGAITNYADSVKGRFTISRDNAKNTVYLQMNSLKPED | | | |
| | TAVYYCAAYEIDWPYFGYWGQGTQVTVSS | | | |
| 5 | QVQLQESGGGLVQAGGSLRLSCAASGYIFVGTYMGWYRQAPGKE | 22 | 638 | |
| | REFVAGIASGSSTYYADSVKGRFTISRDNAKNTVYLQMNSLKPEDT | | | |
| | AVYYCAVFYGDQDDSNILYTYWGQGTQVTVSS | | | |
| 6 | QVQLQESGGGLVQAGGSLRLSCAASGSISNDTPMGWYRQAPGKE | 23 | 468 | |
| | RELVAAISYGASTYYADSVKGRFTISRDNAKNTVYLQMNSLKPED | | | |
| | TAVYYCAADVAIYYWLLYWGQGTQVTVSS | | | |
| 7 | QVQLQESGGGLVQAGGSLRLSCAASGSISTGDGMGWYRQAPGKE | 24 | 393 | |
| | REFVAAISTGAITYYADSVKGRFTISRDNAKNTVYLQMNSLKPEDT | | | |
| | AVYYCAVEAYVDRRFAYYYNYWGQGTQVTVSS | | | |
| 8 | QVQLQESGGGLVQAGGSLRLSCAASGNIFGYDAMGWYRQAPGKE | 25 | 359 | |
| | RELVAGIDIGAITYYADSVKGRFTISRDNAKNTVYLQMNSLKPEDT | | | |
| | AVYYCAAVDYGESYTYSAHSYWGQGTQVTVSS | | | |
| 9 | QVQLQESGGGLVQAGGSLRLSCAASGYIFDVDYMGWYRQAPGKE | 26 | 343 | |
| | RELVAAIDGGTTTYYADSVKGRFTISRDNAKNTVYLQMNSLKPED | | | |
| | TAVYYCAVAVLYDAYPADDLFLTLYYWGQGTQVTVSS | | | |
| 10 | QVQLQESGGGLVQAGGSLRLSCAASGTIFGGVDMGWYRQAPGKE | 27 | 321 | |
| | REFVAAIAAGATTYYADSVKGRFTISRDNAKNTVYLQMNSLKPED | | | |
| | TAVYYCAAFDYDLAWTYFDLDYWGQGTQVTVSS | | | |
| 11 | QVQLQESGGGLVQAGGSLRLSCAASGTIFTVGYMGWYRQAPGKE | 28 | 308 | |
| | REFVAAIDNGANTYYADSAKGRFTISRDNAKNTVYLQMNSLKPED | | | |
| | TAVYYCAVTHGPYSVLGYWGQGTQVTVSS | | | |
| 12 | QVQLQESGGGLVQAGGSLRLSCAASGSIFESGYMGWYRQAPGKE | 29 | 216 | |
| | REFVAAISGGTITYYADSVKGRFTISRDNAKNTVYLQMNSLKPEDT | | | |
| | AVYYCAAYGVASQEAVYYLDYWGQGTQVTVSS | | | |
| 13 | QVQLQESGGGLVQAGGSLRLSCAASGTISIYQAMGWYRQAPGKER | 7 | 211 | TEVp- |
| | ELVAAIAEGANTNYADSVKGRFTISRDNAKNTVYLQMNSLKPEDT | | | Nb9 |
| | AVYYCAVAPFYEWYFDYYYIYWGQGTQVTVSS | | | |
| 14 | QVQLQESGGGLVQAGGSLRLSCAASGTISGGGVMGWYRQAPGKE | 30 | 175 | |
| | REFVASIGTGGTTNYADSVKGRFTISRDNAKNTVYLQMNSLKPED | | | |
| | TAVYYCAVAYYYVAYYDTTLDYWGQGTQVTVSS | | | |
| 15 | QVQLQESGGGLVQAGGSLRLSCAASGTISSDGAMGWYRQAPGKE | 31 | 166 | |
| | REFVAAISYGGSTYYADSVKGRFTISRDNAKNTVYLQMNSLKPED | | | |
| | TAVYYCAVAFPVSYYTSATFVYWGQGTQVTVSS | | | |
| 16 | QVQLQESGGGLVQASGSLRLSCAASGTISSQDAMGWYRQAPGKE | 32 | 158 | |
| | REFVATISGGGTTNYADSVKGRFTISRDNAKNTVYLQMNSLKPED | | | |
| | TAVYYCAALGDEAVPTYYIFAYWGQGTQVTVSS | | | |
| 17 | QVQLQESGGGLVQAGGSLRLSCAASGTISPYRWMGWYRQAPGKE | 33 | 110 | |
| | REFVAGIGYGSTTYYADSVKGRFTISRDNAKNTVYLQMNSLKPED | | | |
| | TAVYYCAAHYDVGLNGPYYYTYWGQGTQVTVSS | | | |
| 18 | QVQLQESGGGLVQAGGSLRLSCAASGSISIGSDMGWYRQAPGKER | 34 | 110 | |
| | EFVAAIGNGGSTYYADSVKGRFTISRDNAKNTVYLQMNSLKPEDT | | | |
| | AVYYCAAPSYYGYSLYYWGQGTQVTVSS | | | |
| 19 | QVQLQESGGGLVQAGGSLRLSCAASGTISYGWHMGWYRQAPGKE | 35 | 96 | |
| | RELVATINHGGTTNYADSVKGRFTISRDNAKNTVYLQMNSLKPED | | | |
| | TAVYYCAAEAWTADTTAWDYPYWGQGTQVTVSS | | | |
| 20 | QVQLQESGGGLVQAGGSLRLSCAASGNISSDDLMGWYRQAPGKE | 36 | 95 | |
| | RELVAAIDTGASTYYADSVKGRFTISRDNAKNTVYLQMNSLKPED | | | |
| | TAVYYCAAYTYSQDYWGVLTYVGHGYWGQGTQVTVSS | | | |
| 21 | QVQLQESGGGLVQAGGSLRLSCAASGYISGEDGMGWYRQAPGKE | 37 | 93 | |
| | RELVAGIATGTITYYADSVKGRFTISRDNAKNTVYLQMNSLKPEDT | | | |
| | AVYYCAAEELGWVGIGGSLWYWGQGTQVTVSS | | | |
| 22 | QVQLQESGGGLVQAGGSLRLSCAASGTIFGVEHMGWYRQAPGKE | 38 | 91 | |
| | RELVAGIDSGSTTYYADSVKGRFTISRDNAKNTVYLQMNSLKPED | | | |
| | TAVYYCAASYPLHYLDTWWFIYWGQGTQVTVSS | | | |
| 23 | QVQLQESGGGLVQAGGSLRLSCAASGYISDPVAMGWYRQAPGKE | 11 | 90 | TEVp- |
| | LELVAAIDYGTTTNYADSVKGRFTISRDNAKNTVYLQMNSLKPED | | | Nb5 |
| | TAVYYCAVEAADYLFGYGYYAYWGQGTQVTVSS | | | |
| 24 | QVQLQESGGGLVQAGGSLRLSCAASGYISNGDYMGWYRQAPGKE | 39 | 79 | |
| | REFVAGIGYGATTYYADSVKGRFTISRDNAKNTVYLQMNSLKPED | | | |
| | TAVYYCAAYTYSQDYYDSGDAYTLIYWGQGTQVTVSS | | | |
| 25 | QVQLQESGGGLVQAGGSLRLSCAASGTIFTYLYMGWYRQAPGKE | 40 | 75 | |
| | RELVASIGNGASTYYADSVKGRFTISRDNAKNTVYLQMNSLKPED | | | |
| | TAVYYCAALSFQEYPIADYFYYWGQGTQVTVSS | | | |
| 26 | QVQLQESGGGLVQAGGSLRLSCAASGYIFYSYAMGWYRQAPGKE | 41 | 75 | |
| | RELVASIGDGASTYYADNVKGRFTISRDIAKNTVYLQMNSLKPEDT | | | |
| | AVYYCAVSYDLDLYGLYPYEYWGQGTQVTVSS | | | |
| 27 | QVQLQESGGGLVQAGGSLRLSCAASGSIFSYYAMGWYRQAPGKE | 42 | 73 | |
| | REFVAGIANGAITNYADSVKGRFTISRDNAKNTVYLQMNSLKPED | | | |
| | TAVYYCAAYEIDWPYFGYWGQGTQVTVSS | | | |
| 28 | QVQLQESGGGLVQAGGSLRLSCAASGNIFRPGTMGWYRQAPGKE | 43 | 68 | |
| | REFVASISLGATTYYADSVKGRFTISRDNAKNTVYLQMNSLKPEDT | | | |
| | AVYYCAAGWGPPFSHSYWGQGTQVTVSS | | | |
| 29 | QVQLQESGGGLVQAGGSLRLSCAASGTIFYEPIMGWYRQAPGKER | 44 | 67 | |
| | ELVAGIDSGSSTYYADSVKGRFTISRDNAKNTVYLQMNSLKPEDT | | | |
| | AVYYCAVDDAWVRYYPGFFYYWGQGTQVTVSS | | | |
| 30 | QVQLQESGGGLVQAGGSLRLSCAASGNISYRGFMGWYRQAPGKE | 45 | 67 | |
| | RELVASIDRGSSTYYADSVKGRFTISRDNAKNTVYLQMNSLKPEDT | | | |
| | AVYYCAAVYSPDNLVLGGLDYWGQGTQVTVSS | | | |
| 31 | QVQLQESGGGLVQAGGSLRLSCAASGTISSGVGMGWYRQAPGKE | 46 | 64 | |
| | RELVATINYGANTNYADSVKGRFTISRDNAKNTVYLQMNSLKPED | | | |
| | TAVYYCAVHYYTTSGAVVYFDYWGQGTQVTVSS | | | |
| 32 | QVQLQESGGGLVQAGGSLRLSCAASGYISPYLVMGWYRQAPGKE | 47 | 62 | |
| | REFVAAIAFGTITNYADSVKGRFTISRDNAKNTVYLQMNSLKPEDT | | | |
| | AVYYCAAIADAGGPIIGDFYYWGQGTQVTVSS | | | |
| 33 | QVQLQESGGGLVQPGGSLRLSCTASGFTLDYYNIGWFRQAPGKER | 48 | 61 | |
| | ERVSCISSSGGRTNYADSVKGRFTISRDNAKNTVYLQMNSLKPEDT | | | |
| | GVYYCAHCMATTEGYEYDYWGQGTQVTVSS | | | |
| 34 | QVQLQESGGGLVQAGGSLRLSCAASGSIFGSAYMGWYRQAPGKE | 49 | 59 | |
| | REFVAAIDYGSTTYYADSVKGRFTISRDNAKNTVYLQMNSLKPED | | | |
| | TAVYYCAAHSYRELYYYGDYPYWGQGTQVTVSS | | | |
| 35 | QVQLQESGGGLVQAGGSLRLSCAASGNISYFIKMGWYRQAPGKER | 50 | 57 | |
| | ELVATIGEGGITYYADSVKGRFTISRDNAKNTVYLQMNSLKPEDTA | | | |
| | VYYCAADNVIIAPYFWAGYYVLDYWGQGTQVTVSS | | | |
| 36 | QVQLQESGGGLVQAGGSLRLSCAASGYIFTSDEMGWYRQAPGKE | 51 | 56 | |
| | RELVAGIGDGGSTYYADSVKGRFTISRDNAKNTVYLQMNSLKPED | | | |
| | TAVYYCAAFGAYSAHNAAIFFYWGQGTQVTVSS | | | |
| 37 | QVQLQESGGGLVQAGGSLRLSCAASGTISRGSAMGWYRQAPGKE | 52 | 55 | |
| | REFVAGITEGAITYYADSVKGRFTISRDNAKNTVYLQMNSLKPEDT | | | |
| | AVYYCAAWTVNNVGYGYWGQGTQVTVSS | | | |
| 38 | QVQLQESGGGLVQAGGSLRLSCAASGSISDDDAMGWYRQAPGKE | 53 | 55 | |
| | RELVAAINYGSTTYYADSVKGRFTISRDNAKNTVYLQMNSLKPED | | | |
| | TAVYYCAADSETDDDGGGDHVYWGQGTQVTVSS | | | |
| 39 | QVQLQESGGGLVQAGGSLRLSCAASGTISPPTFMGWYRQAPGKER | 54 | 54 | |
| | EFVAGIANGGSTYYADSVKGRFTISRDNAKNTVYLQMNSLKPEDT | | | |
| | AVYYCAADPLARADYRYWGQGTQVTVSS | | | |
| 40 | QVQLQESGGGLVQAGGSLRLSCAASGNIFR VREMGWYRQAPGKE | 55 | 50 | |
| | REFVAGIAGGSNTYYADSVKGRFTISRDNAKNTVYLQMNSLKPED | | | |
| | TAVYYCAVGDDVHYSYYYEFPYWGQGTQVTVSS | | | |
| 41 | QVQLQESGGGLVQAGGSLRLSCAASGYISDSGAMGWYRQAPGKE | 56 | 49 | |
| | RELVAAIGGGGSTYYADSVKGCFTISRDNAKNTVYLQMNSLKPED | | | |
| | TAVYYCAVVDAYRDSDYTVLLYWGQGTQVTVSS | | | |
| 42 | QVQLQESGGGLVQAGGSLRLSCAASGNISVADWMGWYRQAPGK | 57 | 49 | |
| | ERELVAAIDAGSITYYADSVKGRFTISRDNAKNTVYLQMNSLKPED | | | |
| | TAVYYCAAFQKVYQSFGYTFGYWGQGTQVTVSS | | | |
| 43 | QVQLQESGGGLVQAGGSLRLSCAASGYIFYYDGMGWYRQAPGKE | 58 | 48 | |
| | RELVAAIDDGGITNYADSVKGRFTISRDNAKNTVYLQMNSLKPED | | | |
| | TAVYYCAVYWLGDVLYQYWGQGTQVTVSS | | | |
| 44 | QVQLQESGGGLVQAGGSLRLSCAASGYIFGGGPMGWYRQAPGKE | 59 | 48 | |
| | REFVATIDPGGITYYADSVKGRFTISRDNAKNTVYLQMNSLKPEDT | | | |
| | AVYYCAAYEIDWPYFGYWGQGTQVTVSS | | | |
| 45 | QVQLQESGGGLVQAGGSLRLSCAASGSIFNDVHMGWYRQAPGKE | 60 | 47 | |
| | REFVSAIASGGSTYYADSVKGRFTISRDNAKNTVYLQMNSLKPEDT | | | |
| | AVYYCAAYPTAIGYLGYWGQGTQVTVSS | | | |
| 46 | QVQLQESGGGLVQAGGSLRLSCAASGYIFDDADMGWYRQAPGKE | 61 | 42 | |
| | RELVATITTGASTNYADSVKGRFTISRDNAKNTVYLQMNSLKPEDT | | | |
| | AVYYCAAINLEYWIVDYALGYWGQGTQVTVSS | | | |
| 47 | QVQLQESGGGLVQAGGSLRLSCAASGTIFEYLYMGWYRQAPGKE | 62 | 42 | |
| | REFVASIAYGTNTNYADSVKGRFTISRDNAKNTVYLQMNSLKPED | | | |
| | TAVYYCAVIGRNTSRTYIYHYYWGQGTQVTVSS | | | |
| 48 | QVQLQESGGGLVQAGGSLRLSCAASGNISSDDLMGWYRQAPGKE | 63 | 41 | |
| | RELVAAIDTGASTYYADSVKGRFTISCDNAKNTVYLQMNSLKPED | | | |
| | TAVYYCAAYTYSQDYWGVLTYVGHGYWGQGTQVTVSS | | | |
| 49 | QVQLQESGGGLVQAGGSLRLSCAASGSISNDTPMGWYRQAPGKE | 15 | 41 | TEVp- |
| | RELVAAISYGASTYYADSVKGRFTISRDNAKNTVYLQMNSLKPED | | | Nb7 |
| | TAVYYCAAYEIDWPYFGYWGQGTQVTVSS | | | |
| 50 | QVQLQESGGGLVQAGGSLRLSCAASGSIFGWFVMGWYRQAPGKE | 64 | 40 | |
| | REFVAAITYGTTTYYADSVKGRFTISRDNAKNTVYLQMNSLKPED | | | |
| | TAVYYCAAETDFPDYYGYWGQGTQVTVSS | | | |

### Biochemical Characterization and Structural Modeling of TEVp Inhibitory Nbs

To investigate whether TEVp inhibitory Nbs are orthosteric or allosteric inhibitors, we measured the binding affinities of TEVp_Nbs 4, 5, 7, and 9 to deactivated TEVp (dTEVp), and the dTEVp-substrate complex by Biolayer Interferometry (BLI). Deactivated TEVp is a catalytic cysteine-to-alanine mutant (C151A) that retains substrate binding capability and whose X-ray structure overlaps exactly with that of TEVp, allowing assays with a substrate-bound enzyme. We also chose dTEVp rather than TEVp because TEVp, both commercial and in-house grade, is produced as an MBP-[TEV cleavage site]-[HIS tag]-TEVp fusion protein, during which TEVp cleaves itself from the MBP solubilizing protein, allowing it to be purified by IMAC. The residual product, in this case, MBP-ENLYFQ (SEQ ID NO: 79), can remain in the enzyme active site. We reasoned that this issue could inadvertently yield misleading results in binding assays. In fact, this concern was validated in initial Nb binding assays using TEVp, rather than dTEVp, which showed moderate to poor Nb binding (Table 4).

TABLE 4
Binding characterization of isolated TEVp-Nbs with TEVp

| | K_{D} [nM] |
|---|---|
| Nb | TEVp |
| TEVp-Nb4 | 348 ± 14.5 |
| TEVp-Nb9 | 782 ± 37.2 |
| TEVp-Nb5 | 411.7 ± 3.9 |
| TEVp-Nb7 | 2980 ± 106 |

TEVp_Nbs 5, 7, and 9 bind to dTEVp with K_{D} values of 18 nM, 163 nM, and 17 nM, respectively—values in line with their K_{I}—qualifying them as monovalent binders (Table 5). However, they do not bind to the dTEV-substrate complex, supporting a competitive inhibition mechanism.

TABLE 5
Binding characterization of isolated TEVp-Nbs

| | K_{D} [nM] | K_{D} [nM] | K_{D} [nM] |
|---|---|---|---|
| Nb | dTEVp | dTEVp + Sub | TVMVp |
| TEVp-Nb4 | 2050 ± 130 | 66 ± 13.5 | N/D |
| TEVp-Nb9 | 18 ± 2.2 | N/D | N/D |
| TEVp-Nb5 | 163 ± 21.1 | N/D | N/D |
| TEVp-Nb7 | 17 ± 2.6 | N/D | N/D |

N/D: Not detectable

We generated Alphafold3 models of Nbs 5, 7, and 9 with dTEVp. The models averaged acceptable predicted template modeling (pTM) scores (>0.5), but low interface predicted template modeling (ip™) values (<0.5). This means that the overall model might be similar to the true structure but with low confidence in the relative positions of the interacting residues. Nonetheless, models of Nb5, 7, and 9 bound to dTEVp all corroborate active-site inhibition. Overall, the long CDR3s (variable lengths ≥11) of Nbs 5, 7, and 9 engage TEVp's active site in a non-substrate-like manner, significantly disrupting the positioning and, in some cases, the secondary structures of the highly flexible C-terminus of TEVp (Gln195-Pro226), which includes S2-S5 pocket residues. Additionally, parts of the CDR3s of Nb7 and Nb9 form parallel β-sheets with those of TEVp's S2 subsite, likely contributing to stabilizing the large structural distortions.

Nb4 is an Uncompetitive Inhibitor that Engages a Positively Charged Pocket Outside TEVp's S2 Subsite.

While Nbs 5, 7, and 9 were identified as active-site inhibitors, BLI results suggest that TEV-Nb4 functions as an uncompetitive inhibitor. TEVp-Nb4 has a K_{I} of 6.7 nM but binds weakly to dTEVp alone (~2 μM). However, TEVp-Nb4 binding affinity increases significantly to 66 nM in the presence of the dTEVp-substrate complex (Table 5). AlphaFold3 models of TEVp-Nb4 bound to the dTEVp-substrate complex yielded acceptable pTM (0.62) and ip™ (0.63) values, providing preliminary insights into this novel binding mechanism. The highly charged CDR3 of TEVp-Nb4 (VDRGDTGPYP; SEQ ID NO: 6) interacts with a positively charged shallow depression on the opposite side of the S2 subsite through an intricate network of hydrogen bonds and hydrophobic interactions. Specifically, Asp101 of Nb4 forms strong hydrogen bonds with Leu210 and Trp211 of TEVp while also engaging in hydrophobic interactions with Val209. Hydrogen bonding between Thr102 of Nb4 and Asp78 of TEVp anchors the CDR3 against one of the pocket's flexible loops (Leu76-Met82). Meanwhile, a Glyl03^{Nb4}-Trp202^{TEVp} hydrogen bond, along with a network of interactions between Asn205^{TEVp}, Glyl00^{Nb4}, and Asp98^{Nb4}, stabilizes the CDR3 against the opposite, lengthy, flexible loop (Ala194-Ser208). Additionally, the backbone carboxyl of Asp101 forms an H-bond with the amino side chain of Lys45 of TEVp, further securing the CDR3 within the shallow pocket. Interestingly, interactions mediated by CDR2 and framework residues further contribute to Nb4's high binding affinity. We hypothesize that, while the conformation of the shallow pocket may be engendered by substrate binding, Nb4 binding likely restricts the movement of the flexible loops and S2 subsite, effectively locking the TEVp-substrate complex into a non-productive state.

Deep Sequencing and Analysis of TEVp_Nb CDR3s Provide Insights into the Molecular Determinants of Inhibition.

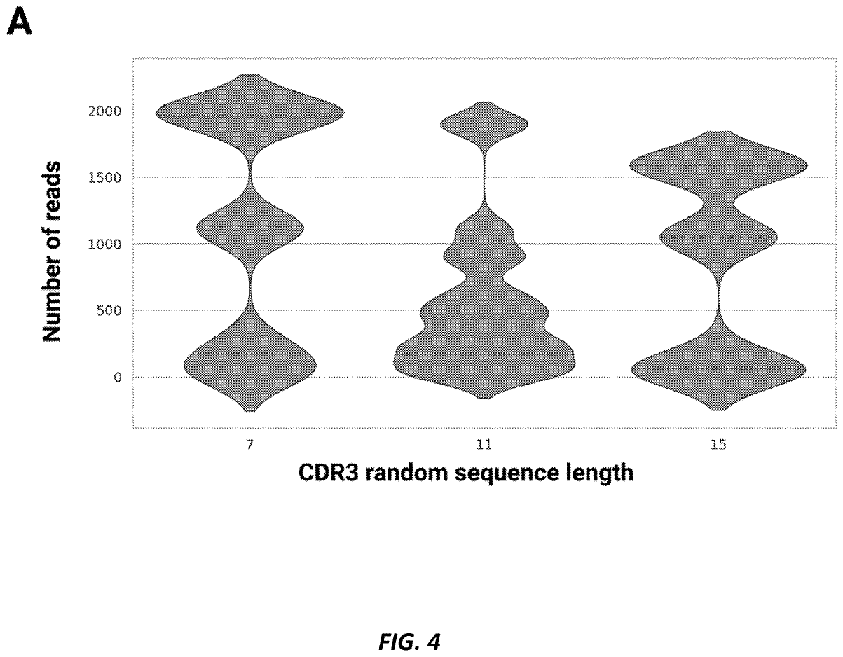

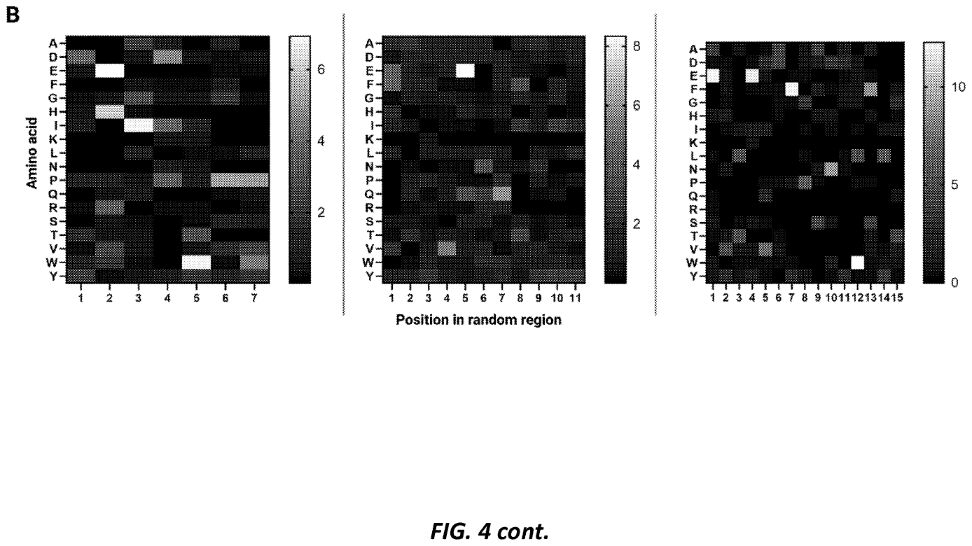

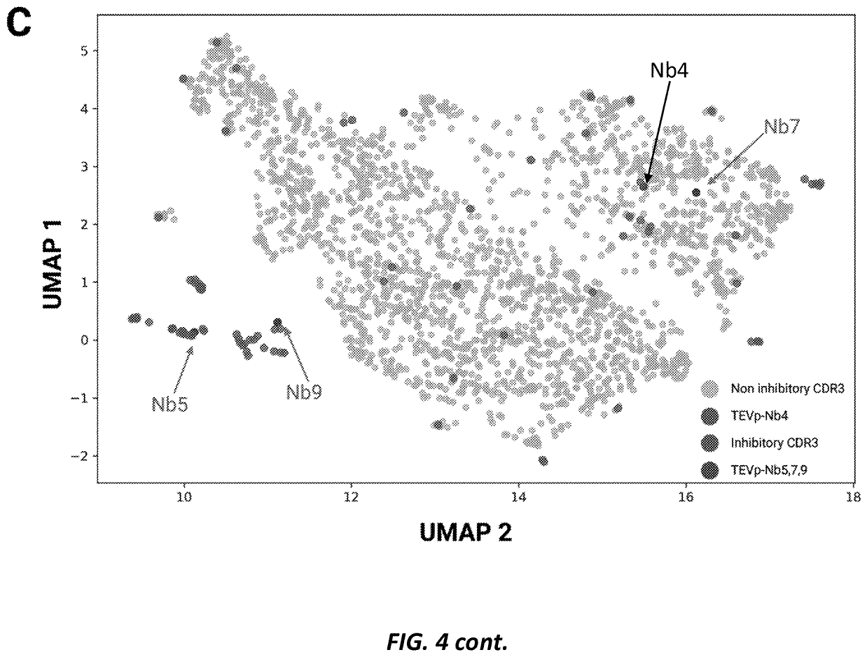

The CDR3 regions of antibodies and nanobodies play a crucial role in determining binding affinity and, in our case, inhibition. Deep sequencing of Nb libraries revealed that while the sorted library contained 34, 138 unique Nb sequences, it included only 1,268 distinct CDR3 sequences, with lengths of 10 (32%), 14 (52%), and 18 (14%) amino acids—mirroring the distribution of the starting library. Highly abundant CDR3s appeared in Nb sequences with high and low read counts, indicating that CDR3s underwent more stringent selection than CDRs 1 and 2. Additionally, while CDR3s of variable length 11 (CDR-11) yielded more inhibitory sequences, CDR-7 inhibitors produced more potent inhibitors (FIG. 4, Panel A), consistent with our identification of TEV-Nb4 as the most effective inhibitor in single-clone screening.

To identify key sequence features of TEVp inhibitory Nbs, we analyzed amino acid frequency enrichments at each position along the CDR3 region by comparing the final sorted library to the original 1-billion-member naïve synthesized library. This analysis revealed both similarities and stark differences in the amino acid composition of inhibitory CDR3s depending on their variable lengths (FIG. 4, Panel B). CDR-11 and CDR-14 sequences displayed similar enrichment patterns, with notable enrichments of Glu at position 1 (P1) and P5, as well as Phe at P7. Co-variance analysis of CDR-11 sequences identified an emerging P5-P7 ENQ motif resembling a TEVp substrate fragment. Interestingly, this motif appears in several highly abundant CDR3s. In contrast to CDR-11 and 14, CDR-7 sequences exhibited high variability across all positions in their variable region. Notable enrichments included Asp at P1 and P4, Glu, His, Arg at P2, Ile at P3, and Pro at P4, P6, and P7. Co-variance analysis of highly abundant sequences identified a P3-P7 EIDW inhibitory motif, which is highly enriched in a dominant CDR3 sequence. One particularly intriguing observation was the enrichment of prolines in CDR-7 sequences. While prolines are rarely found in Nb-binding interfaces, their presence—especially at the borders of CDR3s—limits CDR conformational flexibility and is known to enhance the kinetics and affinity of immunoglobulin interactions. Lastly, investigating the enrichment of Arg at P2 revealed that its enrichment was primarily due to its presence in the highly abundant TEV-Nb4. The CDR3 sequence of TEV-Nb4 diverges from all other Nb inhibitory sequences isolated, suggesting that TEV-Nb4 is a unique Nb, likely binding to a distinct TEVp epitope.

Owing to the diversity of inhibitory CDR3s, and to study differences between inhibitory and non-inhibitory CDR3 sequences, we used EMS-2 and an unsupervised dimensionality reduction with Uniform Manifold Approximation and Projection (UMAP) to embed and visualize Nb sequences in 2D space. UMAP visualization shows that inhibitory Nbs generally clustered away from non-inhibitory ones, suggesting that inhibitory phenotypes may not be solely attributed to single amino acid changes (FIG. 4, Panel C). More importantly, TEV_Nb4 is positioned far from TEV_Nb5, TEV_Nb7, and TEV_Nb9, which bind to the active site, further supporting that Nb4 targets a different TEVp epitope.

### Example 3. Isolation of Human Kallikrein 6 (hK6) Inhibitory Nanobodies

Our proof-of-concept studies with hMMP8 demonstrate that eukaryotic proteases active in the yeast ER can be targeted for inhibitory Nb development. Thus, we next isolated selective inhibitors of human kallikrein 6 (hK6 or KLK6). Human kallikreins (hKs) play key roles in cancer pathophysiology, but their high sequence homology and structural similarity make selective inhibition difficult. Among hKs, hK6 is overexpressed in several cancers, driving tumor invasion and metastasis, and hK6 may also be a viable target to combat neurodegeneration. Currently, no selective small molecule hK6 inhibitors exist, and hK6 is only mildly inhibited by endogenous serine protease inhibitors. Prior efforts focused on reengineering inhibitors based on the amyloid precursor protein Kunitz protease inhibitor (APPI) domain resulted in potent but non-selective clones.

We transformed the Nb library with no ERS into an hK6 yeast reporter strain, as we did for TEVp in Example 2. After four rounds of FACS, library enrichment occurred similar to the TEVp campaign. Confident that hK6 Nbs identified with HARP are inhibitory, we limited our characterization to three hK6-Nb clones. All three hK6-Nbs bound to hK6 with K_{D} values between 200 and 850 nM (Table 6). Interestingly, among the three hK6-Nbs, only hK6-Nb1 was inhibitory.

TABLE 6
Biochemical characterization of hK6-Nbs.

| | | IC₅₀ | K_{D} [nM] | K_{D} [nM] | K_{D} [nM] | K_{D} [nM] | K_{D} [nM] |
|---|---|---|---|---|---|---|---|
| KLK6-Nb-NE | Sequence | [nM] | KLK6 | KLK2 | KLK5 | KLK10 | KLK1 |
| hK6-Nb1 | QVQLQESGGGLVQAGG | 87 ± 54 | | N/D | N/D | N/D | N/D |
| | SLRLSCAASGSIFYYYA | | | | | | |
| | MGWYRQAPGKERELVA | | | | | | |
| | SITAGGITYYADSVKGRF | | | | | | |
| | TISRDNAKNTVYLQMN | | | | | | |
| | SLKPEDTAVYYCAVSHD | | | | | | |
| | SYGNRLDGVTSTHTYW | | 316 ± 8.2 | | | | |
| | GQGTQVTVSS | | | | | | |
| | (SEQ ID NO: 65) (CDR1- | | | | | | |
| | SEQ ID NO: 66; CDR2- | | | | | | |
| | SEQ ID NO: 67; CDR3- | | | | | | |
| | SEQ ID NO: 68) | | | | | | |
| hK6-Nb3 | QVQLQESGGGLVQAGG | N/D | 811 ± 21.9 | not | not | not | not |
| | SLRLSCAASGTISTYGG | | | tested | tested | tested | tested |
| | MGWYRQAPGKERELV | | | | | | |
| | ASIGGGSSTNYADSVKG | | | | | | |
| | RFTISRDNAKNTVYLQ | | | | | | |
| | MNSLKPEDTAVYYCAA | | | | | | |
| | VGATYIIHRYWGQGTQ | | | | | | |
| | VTVSS | | | | | | |
| | (SEQ ID NO: 69) | | | | | | |
| hK6-Nb7 | QVQLQESGGGLVQAGG | N/D | 207 ± 10.5 | not | not | not | not |
| | SLRLSCAASGNIFYGFP | | | tested | tested | tested | tested |
| | MGWYRQAPGKERELV | | | | | | |
| | ATINDGGSTNYADSVK | | | | | | |
| | GRFTISRDNAKNTVYL | | | | | | |
| | QMNSLKPEDTAVYYCA | | | | | | |
| | VGSHTASGDVGYYGSD | | | | | | |
| | FAYWGQGTQVTVSS | | | | | | |
| | (SEQ ID NO: 70) | | | | | | |

N/D: Not detectable.
hK6-Nb1 CDR1, CDR2, and CDR3 are indicated by bold, italics, and underline, respectively.

The unexpected discovery of false positives unveiled a rare failure mode of the HARP approach. We hypothesize that an otherwise non-inhibitory Nb can bind to a protease in a manner that prevents it from binding to the ER receptor. Such an interaction would decrease the enzyme's activity in the ER and could appear as “inhibitory” on flow cytometry. However, we believe such an outcome may not be common and can be mitigated by extending the linker length (i.e., (G₄S) 3 versus only GS in our case) between the protease C-terminus and the ERS.

HK6_Nb1 is a potent and highly selective inhibitor of hK6, with an IC₅₀ of 80 nM. Notably, it fails to bind a panel of other hKs with high sequence similarity (Table 6). Structural analysis based on a highly confident AlphaFold3 model of hK6_Nb1 bound to hK6 (ip™=0.8, pTM=0.82) indicates that hK6_Nb1 functions as an active site-blocking nanobody. Its 18-amino acid CDR3 region is rich in charged and polar residues, including His99, His113, Asp100, Asp107, Asn104, Arg105, Ser97, Ser101, Ser111, Thr110, Thr112, and Thr114. These residues form hydrogen bonds and hydrophobic interactions with the S3 and S1 subsite residues of hK6. Notably, residues 97-102 of the CDR3 form a protruding loop (a convex-shaped paratope) that directly engages the S1 pocket of hK6, while the remaining residues thread along one side of the substrate-binding cleft. This arrangement likely provides structural support and specificity to the highly flexible CDR3 loop. A critical residue in hK6_Nb1 CDR3 is Tyr102, the only tyrosine residue in its CDR3. Tyr102 fits snugly within the S1 pocket of hK6 (residues 189-195, 214-220, and 224-228, along with the catalytic triad). Tyr102 forms a hydrogen bond with the backbone carboxyl group of Ser190 and engages in hydrophobic interactions with Val208 and Gln192. The positioning of Tyr102 in the S1 pocket resembles that of the broad-spectrum active site inhibitor benzamide, suggesting it plays a significant role in hK6 inhibition. Overall, hK6 Nb1 binds to the active site in a non-substrate-like manner, a mechanism that prevents enzymatic cleavage of the inhibitor. This binding mode has been similarly speculated for TEV Nb9 and observed in other systems. Notably, this binding mode is markedly different from that of the APPI inhibitor, which targets the S1′-S3′ subsites of hK6.

### Example 4. Improving Solubility and Stability of Nanobodies with ProteinMPNN

Nb9 was selected as the starting model for redesigning an improved inhibitory nanobody against TEVp. The structure of Nb9 was predicted using AlphaFold3 (AF3). CDR residues were fixed to preserve function, two cysteine residues were fixed to avoid disrupting proper folding, and surface residues (SASA>20) were redesigned, while the remaining backbone residues were kept fixed to maintain the overall fold.

A total of 1,000 sequences were designed using ProteinMPNN Soluble V20 and V30 (at temperatures T=0.1, 0.15, 0.2, 0.25, and 0.3). The top 30 sequences were selected based on AlphaFold2 (AF2)-predicted structural similarity to the parent structure, overall structure score, and ProteinMPNN scores. These top candidates were then filtered using solubility and stability metrics predicted by NetSolP, SoDoPe, TemStaPro, and ESMFold. AlphaFold3 was used to evaluate whether the predicted complex structure of TEVp and the nanobody showed correct CDR positioning and interactions with the TEVp active site.

Finally, the selected candidates were relaxed, and their Rosetta energy (REU) was calculated five times using the Rosetta FastRelax protocol. Designs with lower folded energy than Nb9 were selected. Lower REU values indicate lower internal energy and therefore greater predicted structural stability.

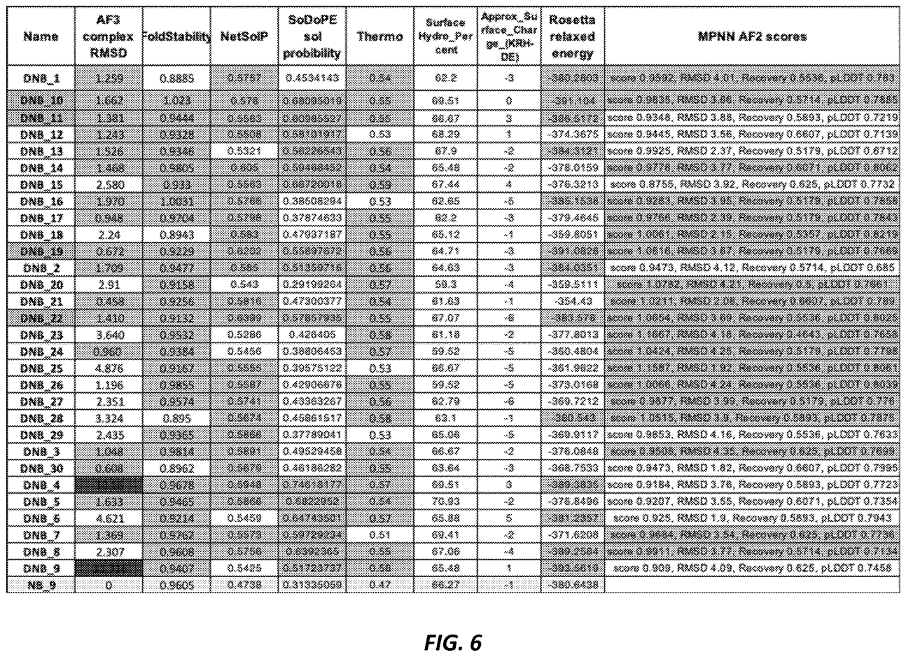

Selection criteria for improved nanobodies included: (i) ProteinMPNN score >0.95; (ii) AF2 Multimer RMSD<5 A; (iii) AF3 multimer RMSD<2 A; (iv) NetSolP-1.0>0.55; (v) SoDoPe prob>50; (vi) ESMFold stability>0.90; and (vii) TemStaPro>54. Results for the 30 candidate nanobodies are shown in FIG. 6. Successfully optimized candidates according to the criteria above were selected and are presented in Table 7 below.

TABLE 7
Solubility and stability optimized TEVp-Nbs

| | | SEQ |
|---|---|---|
| | | ID |
| Nb Name | Sequence | NO |
| DNB_10 | EVKLKESGGGVVKEGGSLTLSCKASGTISIYQAGWYRQ | 85 |
| | APGKDRELVAAIAEGANTNYHPKVEGRFKISRDDSKNT | |
| | VYLEKDLKPEDTATYYCAVAPFYEWYFDYYYIYWGEGT | |
| | KVTVLP | |
| DNB_11 | EVKLTESGGGVVKEGGSLTLSCVASGTISIYQAGWYRK | 86 |
| | APGKPRELVAAIAEGANTNYHDSVKGRFEISRDDSKST | |
| | VFLKNNLKPEDTATYYCAVAPFYEWYFDYYYIYWGKGT | |
| | KVTVKP | |
| DNB_19 | AVTLTESGGGTVKEGGSITLTCTASGTISIYQAGWYRQ | 87 |
| | APGKPRELVAAIAEGANTNYAEKVQGRFKISRDDAAQT | |
| | VTLDTNLKVEDTAVYYCAVAPFYEWYFDYYYIYWGAGT | |
| | RVTVEP | |
| DNB_22 | AVVLTESGGGTVKEGESITLTCKASGTISIYQAGWYRT | 88 |
| | APGEERELVAAIAEGANTNYAERVKGRFTISRDDANNT | |
| | VYLNNNLKVEDTALYYCAVAPFYEWYFDYYYIYWGSGT | |
| | QVIVEE | |

CDR1, CDR2, and CDR3 are indicated by bold, italics, and underline, respectively.

### Materials and Methods

### System Construction

Inhibitor plasmid-A receiver plasmid, pN3 (Table 8), was designed to contain a Cen6/ARS copy number gene, the hygromycin resistance (HygroR) selection marker, and unique cute sites (HindIII and XhoI) for ease of cloning. Using the unique cut sites, pN3 was linearized and was then assembled with the Nb-containing insert (with homology regions to the 5′ and 3′ ends of the linear plasmid) using Gibson Assembly. The pN3-Nb assemblies (Table 8) were transformed into competent Escherichia coli (E. coli) (NEB #C2984H), purified (QIAGEN #27106), and sent for sequencing to confirm correct assembly (Plasmidsaurus, standard plasmid).

TABLE 8
Plasmids referenced in Examples.

| | | |
|---|---|---|
| Strain or | | Plasmid Map |
| Plasmid | Key Characteristics | (if applicable) |

Strains

| | |
|---|---|
| LLE | S. cerevisiae |
| | MATa AGA1::GAL1-AGA1::URA3 ura3-52 |
| | trp1 leu2-delta200::pACT1-LexA-hER- |
| | haB112-ADH1t_pTEF1-ble-TEFt his3- |
| | delta200 pep4::HIS3 prbd1.6R can1 GAL |
| LLE-pY3-LYS- | Integration Site: LYS2 |
| TEVp-FF | Yeast Marker: Leu2 |
| | Protease Promoter: pGAL1-10 |
| | Protease: TEVp |
| | Protease ERS: FEHDEL (SEQ ID NO: 2) |
| | Substrate Promoter: pGAL1-10 |
| | Substrate: ENLYFQS (SEQ ID NO: 78) |
| | Substrate ERS: FEHDEL (SEQ ID NO: 2) |
| LLE-pY2- | Integration Site: MET15 |
| MET- | Yeast Marker: Leu2 |
| TEVsubstrate- | Substrate Promoter: pGAL1 |
| FEH | Substrate: ENLYFQS (SEQ ID NO: 78) |
| | Substrate ERS: FEHDEL (SEQ ID NO: 2) |

Plasmids

| | | |
|---|---|---|
| pN3 | Promoter: pGAL1 | benchling.com/s/seq- |
| | E. coli Marker: AmpR | SFBATiwJUwbjJzeOdINH?m = slm- |
| | Yeast Marker: HygroR | q7p9tIrfJhJD3K6QqrDD |
| | Copy Number: Cen6/ARS | |
| pY3-LYS-IV | Integration Site: LYS2 | benchling.com/s/seq- |
| | Yeast Marker: Leu2 | WFK228gqqGFB7y7hjGHe?m = slm- |
| | Protease Promoter: pGAL1-10 | 1xOs5ZvtCMjLeJXS8KY1 |
| | Substrate Promoter: pGAL1-10 | |
| pN3-Nb14 | Nb Insert: Nb14 | benchling.com/s/seq- |
| | Nb Insert ERS: WEHDEL (SEQ ID NO: 1) | YphVNn5J8iiPtqjBzP2c?m = slm- |
| | | JUnicPbFpA8SduF15nEK |
| pN3-Nb14-NE | Nb Insert ERS: NONE | |
| pN3-5vnv | Nb Insert: 5vnv | benchling.com/s/seq- |
| | Nb Insert ERS: WEHDEL (SEQ ID NO: 1) | 8wY9wFGvlFoqmtvBLX7t?m = slm- |
| | | uGRNbtxviS6lXqmr7w57 |
| PY3-MMP8- | Protease Promoter: pGAL1-10 | benchling.com/s/seq- |
| WF | Protease: MMP8p | ZaW46SaHH6HTveq6i9Mq?m = slm- |
| | Protease ERS: WEHDEL (SEQ ID NO: 1) | aXksuZ9BTknB5wtQm9HX |
| | Substrate Promoter: pGAL1-10 | |
| | Substrate: PLGLVA | |
| | Substrate ERS: FEHDEL (SEQ ID NO: 2) | |
| pY3-NbLibRec | Promoter: pGAL1 | benchling.com/s/seq- |
| | E. coli Marker: AmpR | p8oo3ZCT0l1UmuucwoR4?m = slm- |
| | Yeast Marker: TRP | 3So7SifBZJpazMtZQU9O |
| | Copy Number: Cen6/ARS | |
| pET26b-BsaI | | benchling.com/s/seq- |
| | | hefUVTtyLbpSJSYc7kwZ?m = slm- |
| | | BegZ0dRyaGam8Mm2fie3 |
| pET26b-Nb4- | Nb Insert: Nb4-NE | benchling.com/s/seq- |
| NE | | 7zl4FLWMLIUPMvEc9aTf?m = slm- |
| | | K22JrYXrkSAnQ71mEOS4 |
| pET26b-Nb5- | Nb Insert: Nb5-NE | benchling.com/s/seq- |
| NE | | Wu3LgDQDHwfhxGJCDlVo?m = slm- |
| | | UJptLwFwliojcE3vX2mY |
| pET26b-Nb7- | Nb Insert: Nb7-NE | benchling.com/s/seq- |
| NE | | 8AR451w0CnrPMzEXzdyu?m = slm- |
| | | DoOb1D5bZKZr8cYZ6rVY |
| pET26b-Nb9- | Nb Insert: Nb9-NE | benchling.com/s/seq- |
| NE | | zpPuiHwL93DXryerlIHL?m = slm- |
| | | gk2EP3YPBWwDLZEUpwXy |
| pN4 | Promoter: pGAL1 | benchling.com/s/seq- |
| | E. coli Marker: AmpR | TpoHECjJyN5ebG8nfVoH?m = slm- |
| | Yeast Marker: TRP | RYtPQP4wU4r9Apk6Sndu |
| | Copy Number: Cen6/ARS | |

### Proof-of-Concept MMP8 Nb Sort

In preparation, Nb14 (MMP8-inhibiting nanobody) and 5vnv (non-binding, non-inhibitory nanobody) sequences were cloned into a receiver plasmid, pN3, as mentioned above. MMP8 and its canonical substrate were cloned into a pY2 receiver plasmid, with the protease under β-estradiol (BE) induction and the substrate under galactose induction. Once the plasmids were sequenced to confirm correct assembly (Plasmidsaurus, standard plasmid), a double-plasmid transformation was conducted into an engineered S. cerevisiae strain (Table 8), following the protocol provided in the Frozen-EZ Yeast Transformation II Kit (ZymoResearch #T2001). The cells were plated on minimal media (YNB CAA) containing 2% glucose (GLU) and Hygromycin (HYGRO, 200 ug/uL); labeled according to the plasmids transformed into them, MMP8-Nb14 and MMP8-5vnv. Once the plates grew for 2-4 days at 30° C., mature colonies were picked and grown in 1 mL YNB CAA 2% GLU HYGRO at 30° C. for 16-24 hours.

Once saturation was reached, the optical density (OD₆₀₀) was taken to determine the approximate amount of culture required for a starting outgrow to have an OD 600 of 1 in 1 mL media. The calculated culture was inoculated in 1 mL YNB CAA 2% GLU HYGRO and grown to an OD₆₀₀ between 2-4 (30° C., 250 RPM), for approximately 4-6 hours. The OD₆₀₀ of the outgrow was determined and the amount of culture was calculated to have a starting induction OD₆₀₀ of 0.5 in 1 mL media. Required culture volumes were washed in YNB CAA 2% Galactose (GAL) HYGRO media (to remove residual GLU) and then the cell pellets were resuspended in 1 mL YNB CAA 2% GAL HYGRO and 2 μM βE. Induction cultures were grown for 12-16 hours (30° C., 250 RPM), after which the OD₆₀₀ of each culture was taken. From the OD₆₀₀, the amount of culture was calculated to stain 5 million cells with fluorescently labeled antibodies, as well as the volume of each culture (MMP8-Nb14 and MMP8-5vnv) would need to be combined to have a 1:1000 mixture Nb14: 5vnv (totaling to 5 million cells). The three samples, MMP8-Nb14, MMP8-5vnv, and 1:1000, were washed with PBS 0.5% BSA (0.5% BSA, Goldbio 9048-46-8) and then stained with anti-FLAG PE (Biolegend cat #637309) and anti-HA Alexa 647 (Biolegend cat #682404) at RT for 90 minutes. Samples were washed with PBS 0.5% BSA and resuspended for cytometric and FACS (BDFACS Melody, BD Biosciences).

All samples were analyzed using the PE-A (correlating with high anti-FLAG PE signal) and APC-A (correlating with high anti-HA Alexa 647) channels of the BDFACS Melody. To determine the sorting gate strategy, control samples (MMP8-Nb14, MMP8-5vnv) were assayed independently, and a gate was drawn to capture 1% of the cells with the non-inhibitory phenotype, corresponding with 5vnv expression. This gating strategy was selected to ensure that an abundance of the inhibitory phenotype, our positive control (MMP8-Nb14), was present in the gate, alluding to the notion that when the mock “library” is assayed, cells with the inhibitor present would reside in this gate. The 1:1000 mixed population sample was assayed and cells falling in the sorting gate were captured, enriched, and assayed for inhibition enrichment. The cell preparation and cytometric sorts were repeated a total of two rounds, resulting in almost complete retention of the HA tag (correlating to an inhibited proteases) fluorescent epitope (anti-HA Alexa 647). Cells from the enriched population were cultured and the Nb-containing plasmids were purified (QIAGEN #27106) and sent for sequencing to confirm Nb identity (Sanger Sequencing, Genewiz, Azenta Life Sciences).

### Library Construction

DNA preparation—For the first protein scaffold to be screened in HARP, Nbs were selected based on their small size and the ability to purify them relatively easily from E. coli. A starting DNA template was designed with degenerate amino acids within the complementarity-determining regions (CDRs) of a modified 5vnv Nb sequence (FIG. 3, Panel A). The Nb framework, along with additional assembly elements (e.g., homology regions, ER retention signal (ERS), identifiers) (FIG. 3, Panel B) were constructed and synthesized as a double-stranded DNA (dsDNA) pool with Twist Biosciences (variant library synthesis). A nested PCR was conducted to remove the identifier sequences and assemble the library into yeast. A secondary PCR (non-nested) was performed to amplify the library to a final mass of ~15 μg. An additional PCR was required to create the dsDNA required for NbLibrary-NE-TEVp (removal of the FEHDEL [SEQ ID NO: 2] ERS). Table 9 contains primer sequences for the reactions described above.

TABLE 9
Primer sequences for assemblies referenced in Examples.

| Primer Name | Primer Sequence | SEQ ID NO |
|---|---|---|
| TWIST-NbLib- | ACCATGTACCATGTAAACCTGTTATAAC | 71 |
| Nested-F | | |
| TWIST-NbLib- | TGGATCTTAAAAACCAAACGTGG | 72 |
| Nested-R | | |
| Making- | ATGCAGTTACTTCGCTG | 73 |
| NbLib-F | | |
| Making NbLib-R | AGTCGATTTTGTTACATCTACAC | 74 |
| (contains | | |
| FEHDEL [SEQ | | |
| ID NO: 2] ERS) | | |
| Making NbLib-R | TATCAGATCTCGAGCTATTACTAGCCGCG | 75 |
| (without ERS) | GTACCAAGCTTAGCTGCTCACGGTCACCT | |
| | GG | |
| BsaI-NbLib- | TCCTTAGAGGTCTCGGGCAGCGGCAGCCA | 76 |
| NE-PCR-F | GGTGCAGCTGCAGGAAAGC | |
| BsaI-NbLib- | ACATACAGGGTCTCGATGGGCGGCAGCGC | 77 |
| NE-PCR-R | TGCTCACGGTCACCTGGGTG | |

For large Nb library construction, a modified Nb receiver plasmid was required to mitigate antibiotic selection. This receiver plasmid was designed similarly to the previously reported protease-substrate plasmid, pY3. Following the pGAL1 promoter, a multiple cloning site (MCS) and a linker (for additional homology) were added to allow for the insertion of desired genes using restriction enzyme digestion and subsequent ligation. The resulting plasmid, pY3-NbLibRec (Table 8), was transformed into competent Escherichia coli (E. coli) (NEB #C2984H), purified (QIAGEN #27106), and sent for sequencing to confirm correct assembly (Plasmidsaurus, standard plasmid). pY3-NbLibRec was linearized through a large-scale Afl-II and Sal-I restriction enzyme digestion to expose the homology arms and produce the necessary mass of linearized backbone (~15 ug).

Protease yeast strain—Following the integration strategy established previously (see, e.g., Martinusen et al. (2024) Protein Engineering, Design and Selection 37), TEVp, a gift from David Waugh (Addgene plasmid #8827; n2t.net/addgene: 8827; RRID: Addgene_8827), and its canonical substrate (ENLYFQS; SEQ ID NO: 78) were cloned into pY3-LYS-IV plasmid (Table 8). The plasmid was linearized with NotI-HF and was integrated into an engineered S. cerevisiae strain (Table 8) using the lithium acetate (LiAc) transformation protocol established by Gietz and Schiestl (see, e.g., Gietz and Schiestl (2007) Nature Protocols 2(1):31-34). The cells were plated on selection media, SC-LEU, and grow at 30° C. for 2-4 days. Mature colonies were picked and grown in 1 mL selection media at 30° C. for 16-24 hours, until saturated.

Homologous-Directed Recombination (HDR) assembly for the Nb library in yeast-LLE-pY3-LYS-TEVp-FF yeast cells (Table 8) were prepared for electroporation using the protocol reported in Loock et al. (2023) Methods and Protocols 6(5):89. Once prepared, the electro-competent pY3-LYS-TEV-LLE cells (600 uL/aliquot) were kept on ice, along with 1 2-mm electroporation cuvette, for five minutes before 10 ug of both prepared library and linearized backbone were added to the cells. Following a second 10-minute incubator, the cell-DNA mixture was transferred to the cold cuvette and electroporated using the Sc.2 setting on the Bio-Rad MicroPulser (#1652100). Freshly electroporated cells were transferred to a sterile flask containing 12 mL YPD: Sorbitol and incubated for 1 hour (30° C., 250 RPM). The cells were subsequently spun down (4° C., 5 minutes, 500 RCF) and resuspended in a 2-L sterile flask containing 375 mL YNB 2% GLU. Library culture was incubated for 2-3 days (30° C., 250 RPM) and passaged after saturation was reached to an OD₆₀₀ of 0.2 in 500 mL YNB 2% GLU to give a culture 10-20 times larger than the starting library size. Actual library size was determined by serial dilution plating of starting library culture on selection media (YNB CAA 2% GLU) and incubated in a stationary incubator at 30° C. for 2 days. The saturated culture, referred to as NbLibrary-NE-TEVp henceforth, was stored at 4° C. until the screening process.

### Isolating and Purifying Inhibitors Using Fluorescence-Activated Cell Sorting (FACS)

Cell Preparation (NbLibrary-NE-TEVp)—Saturated NbLibrary-NE-TEVp culture (see Methods: Homologous-Directed Recombination (HDR) Assembly of Nb Library in Yeast) was re-inoculated in a sterile 250-mL flask to an OD₆₀₀ of 0.5 in 50 mL selection media (YNB CAA 2% GLU) and grown at 30° C. (250 rpm) until OD₆₀₀ between 2-4 was reached (approximately 5 hours). Once the desired OD₆₀₀ was achieved, the culture was re-inoculated in a sterile 250-mL flask to an OD₆₀₀ of 0.5 in 50 mL induction media (YNB CAA 2% GAL). The induced culture was incubated at 30° C. (250 rpm) for 12-15 hours. Post-induction OD₆₀₀ was taken, and the volume of culture required to contain approximately 10× NbLibrary-NE-TEVp size was calculated and added to 3 mL PBS 0.5% BSA (0.5% BSA, Goldbio 9048-46-8). Cells were washed and stained with fluorescently tagged antibodies (anti-FLAG PE, Biolegend, cat #637309, anti-HA Alexa 647, Biolegend, cat #682404) at a concentration of 10⁵ cells/uL. Staining was done for 90 minutes, in the dark, at RT. Excess antibody was removed with a second PBS 0.5% BSA wash and resuspended for FACS (BDFACS Melody, BD Biosciences).

Cell Preparation (Control Cells)—OD₆₀₀ of the saturated cultures (inoculation from colony, 1 mL YPD, 30° C., 250 RPM) of LLE-pY2-MET-TEVsubstrate-FEH (positive control) and LLE-pY3-LYS-TEVp-FF (negative control) were taken, and volume calculated to re-inoculate to an OD₆₀₀ of 0.5 in 2 mL selection media (SC-URA 2% GLU) were determined. Calculated volumes of each were added to the media and inoculated at 30° C. (250 rpm) for approximately 5 hours, until an OD₆₀₀ between 2-4 was reached. The OD₆₀₀ of the outgrows were taken and volume of cultures were calculated to have an induction starting OD₆₀₀ of 0.5 in 2 mL induction media (SC-URA 2% GAL). Calculated volumes of each were added to the media and inoculated at 30° C. (250 rpm) for 12-15 hours. Post-induction OD₆₀₀ were taken, and the volume of culture required to contain approximately 2 million cells were calculated. The cells were washed in 1 mL PBA 0.5% BSA and resuspended in 50 uL staining mix, containing PBS 0.5% BSA and fluorescently tagged antibodies (anti-FLAG PE, Biolegend-0.5 uL/2 million cells, cat #637309, anti-HA 374 Alexa 647-1 uL/2 million cells, Biolegend, cat #682404). Staining was done for 90 minutes, in the dark, at RT. Excess antibody was removed with a second PBS 0.5% BSA wash and resuspended for FACS (BDFACS Melody, BD Biosciences).

Cytometric Activity Check of Controls—To analyze the control populations using flow cytometry, fluorochrome channels on the BDFACS Melody were selected based on excitation/emission wavelengths of the antibodies used in the cell staining. High display levels of our FLAG epitope tag (stained with anti-FLAG PE) were analyzed using PE-A and high display levels of our HA epitope tag (stained with anti-HA Alexa 647) were analyzed using APC-A. Yeast cell populations were captured via a gate at ≥10⁴ FSC-A and ≥10⁴ SSC-A and were assayed for fluorescence on a PE-A (x) v APC-A (y) dot plot. Displaying cells were captured using a gate at ≥10⁴ PE-A and all ranges of APC-A. FCS Files were obtained and further analyzed using FlowJo (Version 10.9). These populations aim to serve as the phenotypic controls for the FACS rounds to isolate inhibitory Nbs against the target protease (e.g., TEVp).

Selection and Isolation of Inhibitory Nbs Against TEVp via FACS—Using the established controls, an initial Inhibition Sort Gate was drawn around the positive control (LLE-MET-TEVsubstrate-FEH) (Table 8) to simulate the desired phenotypic outcome of this screen (complete inhibition of TEVp) (FIG. 3, Panel C, top left plot). The stained NbLibrary-NE-TEVp cells (10⁸ cells) were screened against the controls and the population (~0.09%) that fell within the Inhibition Sort Gate were isolated and enriched in selection media (YNB CAA 2% GLU, 5 mL, 30° C., 250 RPM, 12-15 hours). This initial enrichment population (ENR1) was prepared for a second round of FACS as previously described, with the amendment that now 2 million cells were required. ENR1 was screened against the controls and the Inhibition Gate was used to isolate a population (~3%) that were collected and enriched in selection media (YNB CAA 2% GLU, 5 mL, 30° C., 250 RPM, 12-15 hours). This second and third enrichments (ENR2 and ENR3) were prepared as mentioned and assayed on the BDFACS Melody, where a population expressing the complete inhibition phenotype desired (high fluorescent signals of anti-FLAG PE and anti-HA Alexa 647). The top 10% of this final population was isolated through a fourth round of FACS (ENR4) and enriched in selection media (YNB CAA 2% GLU, 5 mL, 30° C., 250 RPM, 12-15 hours). Dot plots depicting each round of FACS are presented in FIG. 3.

Cytometric Assay of Single Nb Constructs from Sorted NbLibrary-NE-TEVp-Dilutions of ENR4 were plated using Equation 1 to plate single clones from the sorted NbLibrary-NE-TEVp (SC-NbLibrary-NE-TEVp) at cell densities of 200 and 500 cells/plate.

$$\frac{\# of Cells Collected}{Volume of Collected Cells ( uL )} = \frac{c e l l s}{uL} \qquad ( 1 )$$

These dilutions were plated on selection media (YNB CAA 2% GLU) and incubated at 30° C. for 24-48 hours. Colonies (n=24) containing one Nb plasmid were picked and inoculated in 2 mL selection media and inoculated at 30° C. (250 RPM) for 24 hours. As previously mentioned, these cultures were prepared for cytometric assay (2 million cells). Just as with the FACS isolation, positive (LLE-MET-TEVsubstrate-FEH) and negative (LLE-pY3-LYS-TEVp-FF) controls were used to allow for the quantification of the inhibitory effect of the Nbs (TEVp-Nb 1-24) on TEVp (FIG. 3, Panel D). As previously reported, the ratio of anti-FLAG/anti-HA average fluorescent signals can be quantified to create a fold-change measurement of protease inhibition related to substrate cleavage. The negative control (LLE-pY3-LYS-TEVp-FF) serving as the baseline measurement, with its anti-FLAG/anti-HA average fluorescent ratio normalized to one. Fluorescent ratios from each Nb were calculated and normalized to the baseline anti-FLAG/anti-HA measurement. With this, the degree of inhibition of TEVp equating to the absolute value of the difference between the normalized ratio of the Nb construct and the baseline control, the greater this difference correlating to the strongest inhibitor. Example calculations of TEVp-Nb4 and TEVp-Nb9 degrees of inhibitions, as well as the baseline TEVp activity ratio, are outlined in Equations 2-4.

$$Active TEVp ( baseline acitivity ratio )$$

$$\frac{Mean anti - FLAG Signal of Active TEVp}{Mean anti - HA Signal of Active TEVp} = \frac{35065}{1076} = 32.6 \qquad ( 2 )$$

$$TEVp - Nb 4 Inhibition Ratio ( normalized )$$

$$\frac{( \frac{Mean anti - FLAG Signal of Nb 4 - NE}{Mean anti - HA Signal of Nb 4 - NE} )}{( \frac{Mean anti - FLAG Signal of Active TEVp}{Mean anti - HA Signal of Active TEVp} )} = \frac{\frac{11614}{7351}}{32.6} = 0.05 \qquad ( 3 )$$

$$TEVp - Nb 9 Inhibition Ratio ( normalize )$$

$$\frac{( \frac{Mean anti - FLAG Signal of Nb 4 - NE}{Mean anti - HA Signal of Nb 4 - NE} )}{( \frac{Mean anti - FLAG Signal of Active TEVp}{Mean anti - HA Signal of Active TEVp} )} = \frac{\frac{25416}{7801}}{32.6} = 0.1 \qquad ( 4 )$$

### Plasmid Purification and Sequencing of Single TEVp-Nb Constructs

TEVp-Nb purification plasmids (TEVp-Nb4, TEVp-Nb5, TEVp-Nb7, TEVp-Nb9) were isolated from yeast colonies (1 colony, 1 mL YNB CAA 2% GLU, 30° C., 250 RPM, 12-15 hours) using the QIAGEN Spin Miniprep Kit (#27106, user-established protocol: Isolation of plasmid DNA from yeast PR04.doc Oct-01). The yeast-prepped (YP) plasmids were transformed into DH5α cells (NEB #C2987H), plated on selection media (LB AMP, 100 mg/mL), and grown at 37° C. for 12-16 hours. Once colonies formed, single colonies were picked and inoculated in 5 mL selection media and grown at 37° C. for 12-16 hours (250 RPM). Plasmids were purified using the QIAGEN Spin Miniprep Kit (#27106) and sent for Sanger Sequencing (Genewiz, Azenta Life Sciences). Primers (Table 9) were designed to cover the entire Nb (~500 bp) to determine the amino acid composition of the whole Nb sequence (Table 2), while also adding BsaI restriction enzyme cut sites to each terminus.

### Purifying TEVp-Nb Inhibitors

pET26b_Nb.b201 (a gift from Andrew Kruse (Addgene plasmid #131404; n2t.net/addgene: 131404; RRID:Addgene_131404), was modified to remove Nb.b201, replacing it with a linker region containing two BsaI restriction enzyme cut sites to create pET26b-BsaI (Table 8), which was used as the purification vector. Using a BsaI Golden Gate Assembly, each Nb (TEVp-Nb4, TEVp-Nb5, TEVp-Nb7, TEVp-Nb9) was cloned into pET26b-BsaI, transformed (unpurified) into DH5a cells (NEB #C2987H), plated on selection media (LB KAN, 50 mg/mL), and grown at 37° C. for 12-16 hours. Once colonies formed, single colonies were picked and inoculated in 5 mL selection media and grown at 37° C. for 12-16 hours (250 RPM). Plasmids were purified using the QIAGEN Spin Miniprep Kit (#27106) and sent for full plasmid sequencing (Plasmidsaurus, standard plasmid) to confirm correct assembly.

Following a modified protocol from McMahon et al. (2018) Nature Structural & Molecular Biology 25:289-296, pET26_Nb (Table 8) plasmids were transformed into BL21 (DE3) E. coli cells (NEB #C2527) in preparation of periplasmic protein purification. Purification was conducted via centrifugation of gravity columns containing high density nickel agarose beads (Goldbio #H-350-100) with 100 μL of unpurified lysate and all wash flowthroughs collected for post-purification SDS-PAGE. Purified proteins were eluded in a solution containing 400 mM imidazole and dialyzed in 1 L high-salt buffer for 15 hours (10 kDa MWC SnakeSkin™ Dialysis Tubing, ThermoFisher Scientific #68100). After dialysis, purified Nbs were concentrated using Amicon® Ultra-15 Centrifugal Filters (10 kDa MWC, Millipore Sigma #UFC901024), and concentration was taken using the NanoDrop ONE (ThermoFisher Scientific #ND-ONE-W). Each purified Nb were run on SDS-PAGE for purity confirmation and stored at −80° C. in a 15% glycerol solution.

### TEVp Purification

pRK793, a gift from David Waugh (Addgene plasmid #8827; n2t.net/addgene: 8827; RRID: Addgene_8827) was transformed into BL21 DE3 Codon Plus RIL E. coli (Agilent #230245). Using the Ni-NTA FPLC purification protocol similar to that outlined in Kapust et al. (2001) Protein Engineering 14 (12): 993-1000, a starting culture (IL inoculation volume) was induced to purify the His-tagged TEVp. Purified enzyme was aliquoted and flash frozen, with size and purity confirmation conducted using SDS-PAGE. Activity of TEVp in vitro was confirmed using a FRET kinetic assay of varying concentrations of the purified enzyme compared to a purchased enzyme (NEB TEVp, #P8112S), canonical FRET TEVp substrate (ABZ-SENLYFQSG-Lys (DNP); SEQ ID NO: 81) synthesized by BIoresiliance® (MilliporeSigma) held constant at 20 uM.

### Characterization of Isolated Inhibitors

Deep-Sequencing Analysis of NGS Results-A Jupyter notebook based on Python was developed on HiperGator, the high-performance supercomputer at the University of Florida, to analyze the NGS results. Reverse and forward frames were converted from FASTQ to FASTA format and imported into the Pandas data frame after the sequencing ID was removed. An alignment based on a 12-base mutual region was then performed between the forward reads and the reverse complement of the reverse reads to obtain the full sequence. Next, each round of data was separated using a 3-base unique identifier at the start and end of each NB sequence. The data were translated into amino acid sequences using the Biopython library. After data cleaning, which involved removing unusually short and long reads, the CDRs were extracted from each sequence using the 3 amino acids flanking each side. The number of occurrences of each unique NB sequence and the frequency of each amino acid in each position were calculated for enrichment analysis. Based on 24 inhibitory NB and their corresponding round 4 CDR3 ranks, a dataset of inhibitory (round 4 repeats higher than 40) and non-inhibitory (Repeats in round 4/repeats in round 2<1) was created. CDR3 sequences numerical representation created using the pre-trained Facebook ESM-2B_650Million_parameters language model. To visualize CDR3 sequence similarity, embeddings were reduced to two dimensions using the UMAP-supervised algorithm. The entire NGS data processing, alignment, translation, cleaning, and visualization pipeline has been backed up to GitHub for future use.

FRET-based Assays—For all FRET-based assays, a running buffer (20 mM Hepes (pH 6.5), 120 mM NaCl, 0.4 mM EDTA, 20% glycerol, and 1 mM DTT) was used to prepare the samples.

TEVp activity was assayed in triplicate across a range of concentration gradients (0-50 uM) of the Abz-DNP substrate (Biomatik, Custom Peptide Synthesis). For each experiment, triplicates of 8 50-uL TEVp samples were prepared in the first three columns of a matte black flat bottom 96-well plate (Greiner Bio-One, #655076) at a fixed concentration of 0.4 uM (for a reaction concentration of 0.2 uM in 100 uL). The substrate was prepared in the fifth column, at a volume of 150 uL/well, with each well containing a solution of Abz-DNP substrate at a concentration 2 times that of the reaction concentration. A multichannel pipette was used to add 50 μL of the substrate to each enzyme sample. Fluorescence intensity (320/405 nm) was measured on a BioTek Synergy Neo2 plate reader at 1 read/20 seconds for 30 minutes. Once all assays were completed, the data was exported into GraphPad Prism (10.1.1) for analysis. Slopes were calculated from the linear regions of each time (x-axis) vs. RFU (y-axis) to get the average reaction velocity, which was subsequently plotted against substrate concentration (on the x-axis). Average K_{M} and vₘₐₓ were extrapolated using a nonlinear regression Michaelis-Menten curve fit, with the error based on the standard deviation of each K_{M} calculated for each set of substrate concentrations.

Nb-mediated inhibition of TEVp activity was assayed in triplicate across a concentration gradient of Nb for each inhibition assay, while the enzyme and substrate concentrations were held constant at 0.2 and 20 uM, respectively. For each experiment, triplicates of 8 50-uL TEVp samples were prepared in the first three columns of a matte black flat bottom 96-well plate (Greiner Bio-One, #655076) at a fixed concentration of 0.4 uM (for a reaction concentration of 0.2 uM in 100 uL). The Nb being assayed in the experiment (one Nb was tested at a time) was prepared in the fourth column of the plate using a 2-fold dilution down the column, each well with a final volume of 100 uL. Using a multichannel pipette, 25 uL of each Nb dilution was added to the TEVp samples, creating triplicates of each enzyme-Nb sample (final Nb concentration for a 100 uL reaction is ¼ the dilution concentration). The enzyme-Nb samples were incubated in the dark for 30 minutes at RT. The substrate was prepared in the fifth column, at a volume of 100 uL/well, with each well containing a solution of Abz-DNP substrate at a concentration of 80 uM, 4 times that of the reaction concentration. Once the incubation period was completed, a multichannel pipette was used to add 25 uL of the substrate to each enzyme-Nb sample. Fluorescence intensity (320/405 nm) was measured on a BioTek Synergy Neo2 plate reader at 1 read/20 seconds for 30 minutes. Once all assays were completed, the data was exported into GraphPad Prism (10.1.1) for analysis. Slopes were calculated from the linear regions of each time (x-axis) vs. RFU (y-axis) to get the average reaction velocity, which was then normalized to the slope of a TEVp-substrate sample (no Nb) at the reaction concentrations. The normalized slopes were converted to a percentage, with the TEVp-substrate sample being 100% active, and the activity percentages were plotted against Nb concentration (on the x-axis). Average IC₅₀ values for each Nb were extrapolated using a nonlinear regression [inhibitor] v. response variable slope (four parameters) curve fit, with the error based on the standard deviation of each IC₅₀ calculated for each sample within the triplicate. Inhibition constants, K_{I}, for each Nb, were calculated using Equation 5, and the error was subsequently calculated for each Nb using Equation 6.

$${K}_{I , avg} = \frac{IC {50}_{avg}}{1 + \frac{[ S ]}{{K}_{M , avg}}} \qquad ( 5 )$$

$${K}_{I , error} = \frac{1}{{K}_{I , avg}} \sqrt{(} {( IC {50}_{error} )}^{2} + {( {K}_{M , error} )}^{2} ) \qquad ( 6 )$$

This assay was conducted as described for all FRET-based inhibition assays.

Biolayer Interferometry (BLI) Binding Assays—For each BLI assay, PBST (0.05% Tween) was used as the neutral/running buffer and Regeneration Buffer (No Salt) (GatorBio, #120063) was used as the regeneration buffer. To maintain consistent probe batches, a single set of probes was used for all the BLI experiments.

The binding affinity of each Nb-NE to TEVp was determined via a standard BLI Kinetic (K) Assay conducted on the GatorBio GatorPlus BLI. Using anti-VHH probes (GatorBio, #160032), each assay began with saturating the probes with Nb at a constant concentration of 34.4 nM for two minutes between the probes were introduced to varying concentrations of TEVp for 300-second association and dissociation steps. Curve binding of the association and disassociation curves were calculated to an R²>0.9 to obtain the kₒₙ and k_{off} values, and thus K_{D} values for each Nb, with the errors (G) calculated using Equation 7.

$${σ}_{a v g} = {K}_{D , avg} \times ( \sqrt{{( \frac{{σ}_{kon , avg}}{{k}_{on , avg}} )}^{2} + {( \frac{{σ}_{koff , avg}}{{k}_{off , avg}} )}^{2}} ) \qquad ( 7 )$$

This assay was conducted as described for all BLI binding assays.

We claim:

1. A method of inhibiting tobacco etch virus protease (TEVp), comprising contacting the TEVp with a nanobody inhibitor.

2. The method of claim 1, wherein the nanobody inhibitor comprises a sequence selected from the group consisting of SEQ ID NOS: 3, 7, 11, 15, 19-64, and 85-88.

3. The method of claim 1, wherein the nanobody inhibitor comprises:
(i) a sequence at least 95% identical to the sequence set forth in SEQ ID NO: 3;
(ii) a sequence at least 95% identical to the sequence set forth in SEQ ID NO: 7;
(iii) a sequence at least 95% identical to the sequence set forth in SEQ ID NO: 11;
(iv) a sequence at least 95% identical to the sequence set forth in SEQ ID NO: 15;
(v) a sequence at least 95% identical to the sequence set forth in SEQ ID NO: 85;
(vi) a sequence at least 95% identical to the sequence set forth in SEQ ID NO: 86;
(vii) a sequence at least 95% identical to the sequence set forth in SEQ ID NO: 87; or
(viii) a sequence at least 95% identical to the sequence set forth in SEQ ID NO: 88.

4. The method of claim 1, wherein the nanobody inhibitor comprises:
(i) the CDR3 sequence set forth in SEQ ID NO: 6;
(ii) the CDR3 sequence set forth in SEQ ID NO: 10;
(iii) the CDR3 sequence set forth in SEQ ID NO: 14; or
(iv) the CDR3 sequence set forth in SEQ ID NO: 18.

5. The method of claim 1, wherein the nanobody inhibitor has an IC₅₀ of less than 500 nM.

6. The method of claim 5, wherein the nanobody inhibitor has an IC₅₀ of less than 50 nM.

7. The method of claim 1, wherein the nanobody inhibitor has a K₁ of less than 150 nM.

8. The method of claim 7, wherein the nanobody inhibitor has a K₁ of less than 25 nM.

9. A method of inhibiting human kallikrein 6 (KLK6), comprising contacting the human KLK6 with a nanobody inhibitor.

10. The method of claim 9, wherein the contacting occurs within a cancer cell, on the surface of a cancer cell, and/or within a tumor cell microenvironment.

11. The method of claim 10, wherein the cancer cell and/or tumor cell microenvironment are in a human subject.

12. The method of claim 9, wherein the nanobody inhibitor comprises a sequence at least 95% identical to the sequence set forth in SEQ ID NO: 65.

13. The method of claim 9, wherein the nanobody inhibitor comprises the CDR3 sequence set forth in SEQ ID NO: 68.

14. The method of claim 9, wherein the nanobody inhibitor:
(i) has a K_{D} of less than 500 nM; and/or
(ii) has an IC₅₀ of less than 100 nM.

15. The method of claim 14, wherein the nanobody inhibitor does not inhibit at least 4 other kallikrein family members.

16. The method of claim 15, wherein the nanobody inhibitor does not inhibit any other kallikrein family members.


SEQUENCE LISTING (88 sequences)

SEQ ID NO: 1 - protein, 6 aa, synthetic construct
WEHDEL

SEQ ID NO: 2 - protein, 6 aa, synthetic construct
FEHDEL

SEQ ID NO: 3 - protein, 118 aa, synthetic construct
QVQLQESGGGLVQAGGSLRLSCAASGTIFHGQFMGWYRQAPGKERELVATIGSGATTYYADSVKGCFTISRDNAKNTVYLQMNSLKPEDTAVYYCAVDRGDTGPYPYWGQGTQVTVSS

SEQ ID NO: 4 - protein, 7 aa, synthetic construct
TIFHGQF

SEQ ID NO: 5 - protein, 12 aa, synthetic construct
LVATIGSGATTY

SEQ ID NO: 6 - protein, 10 aa, synthetic construct
VDRGDTGPYP

SEQ ID NO: 7 - protein, 122 aa, synthetic construct
QVQLQESGGGLVQAGGSLRLSCAASGTISIYQAMGWYRQAPGKERELVAAIAEGANTNYADSVKGRFTISRDNAKNTVYLQMNSLKPEDTAVYYCAVAPFYEWYFDYYYIYWGQGTQVTVSS

SEQ ID NO: 8 - protein, 7 aa, synthetic construct
TISIYQA

SEQ ID NO: 9 - protein, 12 aa, synthetic construct
LVAAIAEGANTN

SEQ ID NO: 10 - protein, 14 aa, synthetic construct
VAPFYEWYFDYYYI

SEQ ID NO: 11 - protein, 122 aa, synthetic construct
QVQLQESGGGLVQAGGSLRLSCAASGYISDPVAMGWYRQAPGKELELVAAIDYGTTTNYADSVKGRFTISRDNAKNTVYLQMNSLKPEDTAVYYCAVEAADYLFGYGYYAYWGQGTQVTVSS

SEQ ID NO: 12 - protein, 7 aa, synthetic construct
YISDPVA

SEQ ID NO: 13 - protein, 12 aa, synthetic construct
LVAAIDYGTTTN

SEQ ID NO: 14 - protein, 14 aa, synthetic construct
VEAADYLFGYGYYA

SEQ ID NO: 15 - protein, 126 aa, synthetic construct
QVQLQESGGGLVQAGGSLRLSCAASGNISSDDLMGWYRQAPGKERELVAAIDTGASTYYADSVKGRFTISCDNAKNTVYLQMNSLKPEDTAVYYCAAYTYSQDYWGVLTYVGHGYWGQGTQVTVSS

SEQ ID NO: 16 - protein, 7 aa, synthetic construct
NISSDDL

SEQ ID NO: 17 - protein, 12 aa, synthetic construct
LVAAIDTGASTY

SEQ ID NO: 18 - protein, 7 aa, synthetic construct
AYTYSQD

SEQ ID NO: 19 - protein, 122 aa, synthetic construct
QVQLQESGGGLVQAGGSLRLSCAASGSIFTDTYMGWYRQAPGKERELVATITPGSSTNYADSVKGRFTISRDNAKNTVYLQMNSLKPEDTAVYYCAAYTYVENQGYYYFAYWGQGTQVTVSS

SEQ ID NO: 20 - protein, 126 aa, synthetic construct
QVQLQESGGGLVQAGGSLRLSCAASGYIFGGGPMGWYRQAPGKEREFVATIDPGGITYYADSVKGRFTISRDNAKNTVYLQMNSLKPEDTAVYYCAAEYTEVDFGSNYWSYGHFYWGQGTQVTVSS

SEQ ID NO: 21 - protein, 118 aa, synthetic construct
QVQLQESGGGLVQAGDSLRLSCAASGSIFSYYAMGWYRQAPGKEREFVAGIANGAITNYADSVKGRFTISRDNAKNTVYLQMNSLKPEDTAVYYCAAYEIDWPYFGYWGQGTQVTVSS

SEQ ID NO: 22 - protein, 122 aa, synthetic construct
QVQLQESGGGLVQAGGSLRLSCAASGYIFVGTYMGWYRQAPGKEREFVAGIASGSSTYYADSVKGRFTISRDNAKNTVYLQMNSLKPEDTAVYYCAVFYGDQDDSNILYTYWGQGTQVTVSS

SEQ ID NO: 23 - protein, 118 aa, synthetic construct
QVQLQESGGGLVQAGGSLRLSCAASGSISNDTPMGWYRQAPGKERELVAAISYGASTYYADSVKGRFTISRDNAKNTVYLQMNSLKPEDTAVYYCAADVAIYYWLLYWGQGTQVTVSS

SEQ ID NO: 24 - protein, 122 aa, synthetic construct
QVQLQESGGGLVQAGGSLRLSCAASGSISTGDGMGWYRQAPGKEREFVAAISTGAITYYADSVKGRFTISRDNAKNTVYLQMNSLKPEDTAVYYCAVEAYVDRRFAYYYNYWGQGTQVTVSS

SEQ ID NO: 25 - protein, 122 aa, synthetic construct
QVQLQESGGGLVQAGGSLRLSCAASGNIFGYDAMGWYRQAPGKERELVAGIDIGAITYYADSVKGRFTISRDNAKNTVYLQMNSLKPEDTAVYYCAAVDYGESYTYSAHSYWGQGTQVTVSS

SEQ ID NO: 26 - protein, 126 aa, synthetic construct
QVQLQESGGGLVQAGGSLRLSCAASGYIFDVDYMGWYRQAPGKERELVAAIDGGTTTYYADSVKGRFTISRDNAKNTVYLQMNSLKPEDTAVYYCAVAVLYDAYPADDLFLTLYYWGQGTQVTVSS

SEQ ID NO: 27 - protein, 122 aa, synthetic construct
QVQLQESGGGLVQAGGSLRLSCAASGTIFGGVDMGWYRQAPGKEREFVAAIAAGATTYYADSVKGRFTISRDNAKNTVYLQMNSLKPEDTAVYYCAAFDYDLAWTYFDLDYWGQGTQVTVSS

SEQ ID NO: 28 - protein, 118 aa, synthetic construct
QVQLQESGGGLVQAGGSLRLSCAASGTIFTVGYMGWYRQAPGKEREFVAAIDNGANTYYADSAKGRFTISRDNAKNTVYLQMNSLKPEDTAVYYCAVTHGPYSVLGYWGQGTQVTVSS

SEQ ID NO: 29 - protein, 122 aa, synthetic construct
QVQLQESGGGLVQAGGSLRLSCAASGSIFESGYMGWYRQAPGKEREFVAAISGGTITYYADSVKGRFTISRDNAKNTVYLQMNSLKPEDTAVYYCAAYGVASQEAVYYLDYWGQGTQVTVSS

SEQ ID NO: 30 - protein, 122 aa, synthetic construct
QVQLQESGGGLVQAGGSLRLSCAASGTISGGGVMGWYRQAPGKEREFVASIGTGGTTNYADSVKGRFTISRDNAKNTVYLQMNSLKPEDTAVYYCAVAYYYVAYYDTTLDYWGQGTQVTVSS

SEQ ID NO: 31 - protein, 122 aa, synthetic construct
QVQLQESGGGLVQAGGSLRLSCAASGTISSDGAMGWYRQAPGKEREFVAAISYGGSTYYADSVKGRFTISRDNAKNTVYLQMNSLKPEDTAVYYCAVAFPVSYYTSATFVYWGQGTQVTVSS

SEQ ID NO: 32 - protein, 122 aa, synthetic construct
QVQLQESGGGLVQASGSLRLSCAASGTISSQDAMGWYRQAPGKEREFVATISGGGTTNYADSVKGRFTISRDNAKNTVYLQMNSLKPEDTAVYYCAALGDEAVPTYYIFAYWGQGTQVTVSS

SEQ ID NO: 33 - protein, 122 aa, synthetic construct
QVQLQESGGGLVQAGGSLRLSCAASGTISPYRWMGWYRQAPGKEREFVAGIGYGSTTYYADSVKGRFTISRDNAKNTVYLQMNSLKPEDTAVYYCAAHYDVGLNGPYYYTYWGQGTQVTVSS

SEQ ID NO: 34 - protein, 118 aa, synthetic construct
QVQLQESGGGLVQAGGSLRLSCAASGSISIGSDMGWYRQAPGKEREFVAAIGNGGSTYYADSVKGRFTISRDNAKNTVYLQMNSLKPEDTAVYYCAAPSYYGYSLYYWGQGTQVTVSS

SEQ ID NO: 35 - protein, 122 aa, synthetic construct
QVQLQESGGGLVQAGGSLRLSCAASGTISYGWHMGWYRQAPGKERELVATINHGGTTNYADSVKGRFTISRDNAKNTVYLQMNSLKPEDTAVYYCAAEAWTADTTAWDYPYWGQGTQVTVSS

SEQ ID NO: 36 - protein, 126 aa, synthetic construct
QVQLQESGGGLVQAGGSLRLSCAASGNISSDDLMGWYRQAPGKERELVAAIDTGASTYYADSVKGRFTISRDNAKNTVYLQMNSLKPEDTAVYYCAAYTYSQDYWGVLTYVGHGYWGQGTQVTVSS

SEQ ID NO: 37 - protein, 122 aa, synthetic construct
QVQLQESGGGLVQAGGSLRLSCAASGYISGEDGMGWYRQAPGKERELVAGIATGTITYYADSVKGRFTISRDNAKNTVYLQMNSLKPEDTAVYYCAAEELGWVGIGGSLWYWGQGTQVTVSS

SEQ ID NO: 38 - protein, 122 aa, synthetic construct
QVQLQESGGGLVQAGGSLRLSCAASGTIFGVEHMGWYRQAPGKERELVAGIDSGSTTYYADSVKGRFTISRDNAKNTVYLQMNSLKPEDTAVYYCAASYPLHYLDTWWFIYWGQGTQVTVSS

SEQ ID NO: 39 - protein, 126 aa, synthetic construct
QVQLQESGGGLVQAGGSLRLSCAASGYISNGDYMGWYRQAPGKEREFVAGIGYGATTYYADSVKGRFTISRDNAKNTVYLQMNSLKPEDTAVYYCAAYTYSQDYYDSGDAYTLIYWGQGTQVTVSS

SEQ ID NO: 40 - protein, 122 aa, synthetic construct
QVQLQESGGGLVQAGGSLRLSCAASGTIFTYLYMGWYRQAPGKERELVASIGNGASTYYADSVKGRFTISRDNAKNTVYLQMNSLKPEDTAVYYCAALSFQEYPIADYFYYWGQGTQVTVSS

SEQ ID NO: 41 - protein, 122 aa, synthetic construct
QVQLQESGGGLVQAGGSLRLSCAASGYIFYSYAMGWYRQAPGKERELVASIGDGASTYYADNVKGRFTISRDIAKNTVYLQMNSLKPEDTAVYYCAVSYDLDLYGLYPYEYWGQGTQVTVSS

SEQ ID NO: 42 - protein, 118 aa, synthetic construct
QVQLQESGGGLVQAGGSLRLSCAASGSIFSYYAMGWYRQAPGKEREFVAGIANGAITNYADSVKGRFTISRDNAKNTVYLQMNSLKPEDTAVYYCAAYEIDWPYFGYWGQGTQVTVSS

SEQ ID NO: 43 - protein, 118 aa, synthetic construct
QVQLQESGGGLVQAGGSLRLSCAASGNIFRPGTMGWYRQAPGKEREFVASISLGATTYYADSVKGRFTISRDNAKNTVYLQMNSLKPEDTAVYYCAAGWGPPFSHSYWGQGTQVTVSS

SEQ ID NO: 44 - protein, 122 aa, synthetic construct
QVQLQESGGGLVQAGGSLRLSCAASGTIFYEPIMGWYRQAPGKERELVAGIDSGSSTYYADSVKGRFTISRDNAKNTVYLQMNSLKPEDTAVYYCAVDDAWVRYYPGFFYYWGQGTQVTVSS

SEQ ID NO: 45 - protein, 122 aa, synthetic construct
QVQLQESGGGLVQAGGSLRLSCAASGNISYRGFMGWYRQAPGKERELVASIDRGSSTYYADSVKGRFTISRDNAKNTVYLQMNSLKPEDTAVYYCAAVYSPDNLVLGGLDYWGQGTQVTVSS

SEQ ID NO: 46 - protein, 122 aa, synthetic construct
QVQLQESGGGLVQAGGSLRLSCAASGTISSGVGMGWYRQAPGKERELVATINYGANTNYADSVKGRFTISRDNAKNTVYLQMNSLKPEDTAVYYCAVHYYTTSGAVVYFDYWGQGTQVTVSS

SEQ ID NO: 47 - protein, 122 aa, synthetic construct
QVQLQESGGGLVQAGGSLRLSCAASGYISPYLVMGWYRQAPGKEREFVAAIAFGTITNYADSVKGRFTISRDNAKNTVYLQMNSLKPEDTAVYYCAAIADAGGPIIGDFYYWGQGTQVTVSS

SEQ ID NO: 48 - protein, 121 aa, synthetic construct
QVQLQESGGGLVQPGGSLRLSCTASGFTLDYYNIGWFRQAPGKERERVSCISSSGGRTNYADSVKGRFTISRDNAKNTVYLQMNSLKPEDTGVYYCAHCMATTEGYEYDYWGQGTQVTVSS

SEQ ID NO: 49 - protein, 122 aa, synthetic construct
QVQLQESGGGLVQAGGSLRLSCAASGSIFGSAYMGWYRQAPGKEREFVAAIDYGSTTYYADSVKGRFTISRDNAKNTVYLQMNSLKPEDTAVYYCAAHSYRELYYYGDYPYWGQGTQVTVSS

SEQ ID NO: 50 - protein, 126 aa, synthetic construct
QVQLQESGGGLVQAGGSLRLSCAASGNISYFIKMGWYRQAPGKERELVATIGEGGITYYADSVKGRFTISRDNAKNTVYLQMNSLKPEDTAVYYCAADNVIIAPYFWAGYYVLDYWGQGTQVTVSS

SEQ ID NO: 51 - protein, 122 aa, synthetic construct
QVQLQESGGGLVQAGGSLRLSCAASGYIFTSDEMGWYRQAPGKERELVAGIGDGGSTYYADSVKGRFTISRDNAKNTVYLQMNSLKPEDTAVYYCAAFGAYSAHNAAIFFYWGQGTQVTVSS

SEQ ID NO: 52 - protein, 118 aa, synthetic construct
QVQLQESGGGLVQAGGSLRLSCAASGTISRGSAMGWYRQAPGKEREFVAGITEGAITYYADSVKGRFTISRDNAKNTVYLQMNSLKPEDTAVYYCAAWTVNNVGYGYWGQGTQVTVSS

SEQ ID NO: 53 - protein, 122 aa, synthetic construct
QVQLQESGGGLVQAGGSLRLSCAASGSISDDDAMGWYRQAPGKERELVAAINYGSTTYYADSVKGRFTISRDNAKNTVYLQMNSLKPEDTAVYYCAADSETDDDGGGDHVYWGQGTQVTVSS

SEQ ID NO: 54 - protein, 118 aa, synthetic construct
QVQLQESGGGLVQAGGSLRLSCAASGTISPPTFMGWYRQAPGKEREFVAGIANGGSTYYADSVKGRFTISRDNAKNTVYLQMNSLKPEDTAVYYCAADPLARADYRYWGQGTQVTVSS

SEQ ID NO: 55 - protein, 122 aa, synthetic construct
QVQLQESGGGLVQAGGSLRLSCAASGNIFRVREMGWYRQAPGKEREFVAGIAGGSNTYYADSVKGRFTISRDNAKNTVYLQMNSLKPEDTAVYYCAVGDDVHYSYYYEFPYWGQGTQVTVSS

SEQ ID NO: 56 - protein, 122 aa, synthetic construct
QVQLQESGGGLVQAGGSLRLSCAASGYISDSGAMGWYRQAPGKERELVAAIGGGGSTYYADSVKGCFTISRDNAKNTVYLQMNSLKPEDTAVYYCAVVDAYRDSDYTVLLYWGQGTQVTVSS

SEQ ID NO: 57 - protein, 122 aa, synthetic construct
QVQLQESGGGLVQAGGSLRLSCAASGNISVADWMGWYRQAPGKERELVAAIDAGSITYYADSVKGRFTISRDNAKNTVYLQMNSLKPEDTAVYYCAAFQKVYQSFGYTFGYWGQGTQVTVSS

SEQ ID NO: 58 - protein, 118 aa, synthetic construct
QVQLQESGGGLVQAGGSLRLSCAASGYIFYYDGMGWYRQAPGKERELVAAIDDGGITNYADSVKGRFTISRDNAKNTVYLQMNSLKPEDTAVYYCAVYWLGDVLYQYWGQGTQVTVSS

SEQ ID NO: 59 - protein, 118 aa, synthetic construct
QVQLQESGGGLVQAGGSLRLSCAASGYIFGGGPMGWYRQAPGKEREFVATIDPGGITYYADSVKGRFTISRDNAKNTVYLQMNSLKPEDTAVYYCAAYEIDWPYFGYWGQGTQVTVSS

SEQ ID NO: 60 - protein, 118 aa, synthetic construct
QVQLQESGGGLVQAGGSLRLSCAASGSIFNDVHMGWYRQAPGKEREFVSAIASGGSTYYADSVKGRFTISRDNAKNTVYLQMNSLKPEDTAVYYCAAYPTAIGYLGYWGQGTQVTVSS

SEQ ID NO: 61 - protein, 122 aa, synthetic construct
QVQLQESGGGLVQAGGSLRLSCAASGYIFDDADMGWYRQAPGKERELVATITTGASTNYADSVKGRFTISRDNAKNTVYLQMNSLKPEDTAVYYCAAINLEYWIVDYALGYWGQGTQVTVSS

SEQ ID NO: 62 - protein, 122 aa, synthetic construct
QVQLQESGGGLVQAGGSLRLSCAASGTIFEYLYMGWYRQAPGKEREFVASIAYGTNTNYADSVKGRFTISRDNAKNTVYLQMNSLKPEDTAVYYCAVIGRNTSRTYIYHYYWGQGTQVTVSS

SEQ ID NO: 63 - protein, 126 aa, synthetic construct; identical to SEQ ID NO: 15

SEQ ID NO: 64 - protein, 118 aa, synthetic construct
QVQLQESGGGLVQAGGSLRLSCAASGSIFGWFVMGWYRQAPGKEREFVAAITYGTTTYYADSVKGRFTISRDNAKNTVYLQMNSLKPEDTAVYYCAAETDFPDYYGYWGQGTQVTVSS

SEQ ID NO: 65 - protein, 126 aa, synthetic construct
QVQLQESGGGLVQAGGSLRLSCAASGSIFYYYAMGWYRQAPGKERELVASITAGGITYYADSVKGRFTISRDNAKNTVYLQMNSLKPEDTAVYYCAVSHDSYGNRLDGVTSTHTYWGQGTQVTVSS

SEQ ID NO: 66 - protein, 8 aa, synthetic construct
GSIFYYYA

SEQ ID NO: 67 - protein, 12 aa, synthetic construct
LVASITAGGITY

SEQ ID NO: 68 - protein, 18 aa, synthetic construct
VSHDSYGNRLDGVTSTHT

SEQ ID NO: 69 - protein, 118 aa, synthetic construct
QVQLQESGGGLVQAGGSLRLSCAASGTISTYGGMGWYRQAPGKERELVASIGGGSSTNYADSVKGRFTISRDNAKNTVYLQMNSLKPEDTAVYYCAAVGATYIIHRYWGQGTQVTVSS

SEQ ID NO: 70 - protein, 126 aa, synthetic construct
QVQLQESGGGLVQAGGSLRLSCAASGNIFYGFPMGWYRQAPGKERELVATINDGGSTNYADSVKGRFTISRDNAKNTVYLQMNSLKPEDTAVYYCAVGSHTASGDVGYYGSDFAYWGQGTQVTVSS

SEQ ID NO: 71 - DNA, 28 bp, synthetic construct
  source 1..28: mol_type = other DNA
accatgtaccatgtaaacctgttataac

SEQ ID NO: 72 - DNA, 23 bp, synthetic construct
  source 1..23: mol_type = other DNA
tggatcttaaaaaccaaacgtgg

SEQ ID NO: 73 - DNA, 17 bp, synthetic construct
  source 1..17: mol_type = other DNA
atgcagttacttcgctg

SEQ ID NO: 74 - DNA, 23 bp, synthetic construct
  source 1..23: mol_type = other DNA
agtcgattttgttacatctacac

SEQ ID NO: 75 - DNA, 60 bp, synthetic construct
  source 1..60: mol_type = other DNA
tatcagatctcgagctattactagccgcggtaccaagcttagctgctcacggtcacctgg

SEQ ID NO: 76 - DNA, 48 bp, synthetic construct
  source 1..48: mol_type = other DNA
tccttagaggtctcgggcagcggcagccaggtgcagctgcaggaaagc

SEQ ID NO: 77 - DNA, 49 bp, synthetic construct
  source 1..49: mol_type = other DNA
acatacagggtctcgatgggcggcagcgctgctcacggtcacctgggtg

SEQ ID NO: 78 - protein, 7 aa, synthetic construct
ENLYFQS

SEQ ID NO: 79 - protein, 6 aa, synthetic construct
ENLYFQ

SEQ ID NO: 80 - protein, 10 aa, synthetic construct
TENLYFQSGK

SEQ ID NO: 81 - protein, 9 aa, synthetic construct
SENLYFQSG

SEQ ID NO: 82 - protein, 117 aa, synthetic construct
QVQLQESGGGLVQAGGSLRLSCAASGYISDAYYMGWYRQAPGKEREFVATITHGTNTYYADSVKGRFTISRDNAKNTVYLQMNSLKPEDTAVYYCAVLETRSYSFRYWGQGTQVTVS

SEQ ID NO: 83 - protein, 120 aa, synthetic construct
QVQLQESGGGLVQPGGSLRLSCTASGFTLDYYNIGWFRQAPGKERERVSCISSSGGRTNYADSVKGRFTISRDNAKNTVYLQMNSLKPEDTGVYYCAHCMATTEGYEYDYWGQGTQVTVS

SEQ ID NO: 84 - protein, 10 aa, synthetic construct
VDRGDTGPYP

SEQ ID NO: 85 - protein, 120 aa, synthetic construct
EVKLKESGGGVVKEGGSLTLSCKASGTISIYQAGWYRQAPGKDRELVAAIAEGANTNYHPKVEGRFKISRDDSKNTVYLEKDLKPEDTATYYCAVAPFYEWYFDYYYIYWGEGTKVTVLP

SEQ ID NO: 86 - protein, 120 aa, synthetic construct
EVKLTESGGGVVKEGGSLTLSCVASGTISIYQAGWYRKAPGKPRELVAAIAEGANTNYHDSVKGRFEISRDDSKSTVFLKNNLKPEDTATYYCAVAPFYEWYFDYYYIYWGKGTKVTVKP

SEQ ID NO: 87 - protein, 120 aa, synthetic construct
AVTLTESGGGTVKEGGSITLTCTASGTISIYQAGWYRQAPGKPRELVAAIAEGANTNYAEKVQGRFKISRDDAAQTVTLDTNLKVEDTAVYYCAVAPFYEWYFDYYYIYWGAGTRVTVEP

SEQ ID NO: 88 - protein, 120 aa, synthetic construct
AVVLTESGGGTVKEGESITLTCKASGTISIYQAGWYRTAPGEERELVAAIAEGANTNYAERVKGRFTISRDDANNTVYLNNNLKVEDTALYYCAVAPFYEWYFDYYYIYWGSGTQVIVEE